(19) United States
(12) Patent Application Publication
(10) Pub. No.: US 2021/0205352 A1
(43) Pub. Date: Jul. 8, 2021

(54) ANTIBIOTIC COMPOSITIONS AND METHODS OF USE

(71) Applicant: The Board of Regents of the University of Oklahoma, Norman, OK (US)

(72) Inventor: Charles V. Rice, Norman, OK (US)

(21) Appl. No.: 17/208,176

(22) Filed: Mar. 22, 2021

Related U.S. Application Data
(63) Continuation-in-part of application No. 16/530,756, filed on Aug. 2, 2019, now Pat. No. 10,953,040.
Application No. 16/530,756 is a continuation of application No. 15/736,675, filed on Dec. 14, 2017, now abandoned.
(60) Provisional application No. 62/180,976, filed on Jun. 17, 2015.

(51) Int. Cl. A61K 31/785; A61K 31/546; A61K 31/545; A61P 31/04; A61K 31/43

(52) U.S. Cl. CPC A61K 31/785; A61K 31/546; Y02A 50/30; A61P 31/04; A61K 31/43; A61K 31/545

(57) ABSTRACT

The present disclosure describes compositions comprising β-lactam antibiotics and branched polyethylenimines (BPEI), having efficacy against various Gram-positive bacteria, for example Gram-positive bacteria having resistance against β-lactam antibiotics. The compositions result in the resensitization of such resistant bacterial strains to traditional antibiotic therapies such as cephem β-lactam antibiotics.

### CROSS-REFERENCE TO RELATED APPLICATIONS

The present application is a continuation-in-part application of U.S. Ser. No. 16/530,756, filed Aug. 2, 2019, which is a continuation application of U.S. Ser. No. 15/736,675, filed Dec. 14, 2017, which is a national stage application of a PCT application having International Application No. PCT/US2016/037799, filed Jun. 16, 2016, which claims the benefit under 35 U.S.C. § 119(e) of U.S. Provisional Patent Application Ser. No. 62/180,976 filed on Jun. 17, 2015. The present application is also a continuation-in-part application of PCT application PCT/US2019/054508, filed Oct. 3, 2019, which claims the benefit under 35 U.S.C. § 119(e) of U.S. Provisional Patent Application Ser. No. 62/747,517, filed on Oct. 18, 2018. The entire contents of each of the above-referenced applications is hereby expressly incorporated herein by reference.

### STATEMENT REGARDING FEDERALLY SPONSORED RESEARCH OR DEVELOPMENT

This invention was made with government support under Grant No. 1R01GM090064-01 awarded by the National Institutes of Health (NIH) and National Institute of General Medical Science (NIGMS). The government has certain rights in the invention.

### BACKGROUND

Resistance of certain bacterial strains to previously-effective antibiotics is a growing global problem. For example, colonies of methicillin-resistant Staphylococcus aureus (MRSA) bacteria invade host tissue to release toxins that cause tissue injury, leading to significant patient morbidity. The patient suffers while numerous first- and second-line antibiotics are prescribed to no avail. This increases the threat of MRSA to public health. Timely MRSA diagnosis and delivering drugs of last resort are essential to prevent mortality. In 2011 for example, MRSA infected 80,500 people and nearly 1 in 7 cases resulted in death (11,300; 14%). While, several antibiotics of last resort (vancomycin, linezolid, daptomycin) are effective at killing MRSA, and there has never been a S. aureus isolate resistant to all approved antibiotics, patients still die from MRSA infections. The reason for this is because the drugs of last resort are given after morbidity from staphylococcal toxins has set in, too late to prevent mortality. Moreover, vancomycin, a primary treatment option after MRSA diagnosis, presents additional barriers of high cost and toxicity. New antibiotics, such as oxadiazoles, tedizolid, and teixobactin, are awaiting FDA approval to meet the critical need for new treatments because S. aureus strains resistant to vancomycin and β-lactams have emerged. New treatment options for MRSA and other bacterial strains which have become resistant to standard β-lactam antibiotics are needed.

Originally acquired exclusively in health care settings, MRSA is now regularly found outside the health care environment for a variety of reasons including drug-resistance, which hinders efforts to develop safe clinical treatments for MRSA infections. Resistance mechanisms include a novel penicillin-binding protein 2a (PBP2a) with a lower affinity for β-lactams than the native penicillin-binding protein 2 (PBP2) protein. β-lactamases, enzymes that degrade β-lactam antibiotics, are also common. Approaches to overcome these resistance factors are efflux-pump inhibitors that increase the intracellular concentration of antibiotics, using β-lactamase inhibitors, or increasing cell wall permeability. Nevertheless, the cell wall remains an especially rich antimicrobial target, containing many components shown to contribute to resistance, such as excess peptidoglycan, techoic acids and novel proteins. While these approaches have shown promise, the presence of side-effects and slow transfer from discovery to clinical usage have failed to stem the rate of MRSA infection and mortality. Community-acquired MRSA is less likely than healthcare-associated MRSA to exhibit multidrug resistance, but resistance to β-lactam antibiotics is a defining characteristic in both cases. New formulations to effectively treat MRSA and other antibiotic-resistant bacteria are urgently needed.

### BRIEF DESCRIPTION OF THE DRAWINGS

Several embodiments of the present disclosure are hereby illustrated in the appended drawings. It is to be noted however, that the appended drawings only illustrate several embodiments and are therefore not intended to be considered limiting of the scope of the present disclosure.

FIG. 1 shows results of an in vitro antimicrobial assay of the effectiveness of BPEI in restoring ampicillin efficacy against MRSA. When the concentration of added ampicillin is constant (9.3 μg/mL), cell growth is not observed when the BPEI concentration is 5.3 μg/mL (right data set, columns with thick diagonal lines). When the BPEI is used without ampicillin, 53 μg/mL of BPEI is required to inhibit cell growth (middle data set, columns with thin diagonal lines). The growth of MRSA was evaluated by measuring the change in OD₆₀₀ after 20 hours. Cell growth in TSB media (control) or media with 1% DMSO are shown by the ΔOD₆₀₀, with 50 μg/mL chloramphenicol as a negative control.

FIG. 2 shows results of an in vitro assay of ampicillin against MRSA. When BPEI (2.7 μg/mL) is added, the MIC for ampicillin is between 12 and 25 μg/mL (checkered columns) while ampicillin without BPEI does not inhibit MRSA below 50 μg/mL concentration (solid columns). The growth of MRSA was evaluated by measuring the change in OD₆₀₀ after 20 hours. Cell growth in TSB without additives is denoted as Control.

FIG. 3 shows results of an in vitro assay of ampicillin against MRSA. When BPEI (2.7 μg/mL) is added, the MIC for ampicillin is approximately 25 μg/mL (checkered columns); without BPEI, the MIC is approximately 50 μg/mL (solid columns). The growth of MRSA was evaluated by measuring the change in OD₆₀₀ after 20 hours. Cell growth in TSB without additives is denoted as Control; addition of 50 μg/mL chloramphenicol provided a negative control.

FIG. 4 show microphotographs of sections having BPEI binding to MRSA and E. coli. Paraformaldehyde-fixed MRSA, imaged by Laser Scanning Confocal Microscopy (LSCM), stained with BPEI-AlexaFluor 488 (A) and 4′,6-diamidino-2-phenylindole (DAPI) (B). The merged image (C) shows BPEI binding to the cell surface but not within the cytoplasm. In contrast, PFA-fixed E. coli stained with BPEI-AlexaFluor 488 (D) and DAPI (E) and merged (F) shows a relatively low affinity between BPEI and E. coli. Scale bar=5 μm.

FIG. 5 shows results of an in vitro ampicillin assay against MRSA. When BPEI (26 μg/mL) is added, the MIC for ampicillin is approximately 1.2 μg/mL. The growth of MRSA was evaluated by measuring the change in OD₆₀₀ after 20 hours. Cell growth in media without additives is denoted as Control, and in media with 1% DMSO as the DMSO bars. The addition of chloramphenicol at 50 μg/mL served as a negative-growth control.

FIG. 6 shows results of an in vitro methicillin assay against MRSA. Addition of 2.65 μg/mL BPEI gives a methicillin MIC of −30 μg/mL (thatched columns), while methicillin by itself does not completely inhibit MRSA growth at 60 μg/mL (striped columns). The growth of MRSA was evaluated by measuring the change in OD₆₀₀ after 20 hours. Cell growth in media without additives is denoted as Control, while addition of 50 μg/mL chloramphenicol serves as a negative control (solid columns).

FIG. 7 shows results of an in vitro amoxicillin assay against MRSA. Addition of 2.65 μg/mL BPEI allows amoxicillin to inhibit MRSA growth at 29 μg/mL (thatched columns); the same level of inhibition requires 58 μg/mL amoxicillin without BPEI (striped columns). MRSA growth was evaluated by measuring the change in OD₆₀₀ after 20 hours. Cell growth in media without additives is denoted as Control; chloramphenicol addition (50 μg/mL) provides a negative control (solid columns).

FIG. 8 shows results of an in vitro chloramphenicol assay against MRSA. MRSA is inhibited with the addition of 5 μg/mL chloramphenicol in conjunction with 2.65 μg/mL BPEI; without BPEI the chloramphenicol MIC is raised to 50 μg/mL. MRSA growth did not differ substantially between normal media (Control), media with 1% DMSO, and media with both DMSO and BPEI.

FIG. 9 shows results of an in vitro vancomycin assay against MRSA. The vancomycin MIC against MRSA does not change between antibiotic alone (striped columns) and addition of 2.65 μg/mL BPEI (thatched columns). The growth of MRSA was evaluated by measuring the change in OD₆₀₀ after 20 hours. Cell growth in media without additives is denoted as Control. Addition of BPEI and DMSO to the media does not hinder MRSA growth, while addition of 50 μg/mL chloramphenicol serves as a negative-growth control (solid columns).

FIG. 10 shows results of an in vitro novobiocin assay against MRSA. Growth of MRSA is not substantially altered from novobiocin alone (striped columns) upon addition of 2.65 μg/mL BPEI (thatched columns). The growth of MRSA was evaluated by measuring the change in OD₆₀₀ after 20 hours. Positive growth controls (in media, media+DMSO, and media+DMSO+BPEI) are shown (solid columns).

FIG. 11 shows results of an in vitro assay of BPEI toxicity against murine cells. Viability of NIH/3T3 mouse fibroblasts over three days was examined through OD₅₇₀ measurements. BPEI has little effect on proliferation or viability of 3T3 murine cells at up to the highest concentration tested, 26.5 μg/mL. Untreated cells are listed as the control; DMSO toxicity is shown for comparison.

FIG. 12 shows results of an in vitro ampicillin assay against Gram-negative E. coli. When BPEI (2.65 μg/mL) is added, the MIC for ampicillin is approximately 3.7 μg/mL (striped columns), identical to that observed with ampicillin alone (checkered columns). The growth was evaluated by measuring the change in OD₆₀₀ after 20 hours. Cell growth in media without additives is denoted as Control, along with growth in media with 1% DMSO and DMSO+BPEI (solid columns).

FIG. 13 shows results of an in vitro BPEI assay against Gram-negative E. coli bacteria. Addition of BPEI does not affect the growth of E. coli, either by itself (striped columns) or in conjunction with 0.37 μg/mL ampicillin (checkered columns). Growth of E. coli was evaluated by measuring the change in OD₆₀₀ after 20 hours. Cell growth in media without additives is denoted as Control and in media with 1% DMSO as DMSO; addition of 50 μg/mL chloramphenicol serves as a negative control (solid columns).

FIG. 14 shows results of an in vitro ampicillin assay against non-resistant S. aureus. When BPEI (2.65 μg/mL) is added, the MIC for ampicillin is approximately 50 ng/mL (striped columns), the same as for ampicillin by itself (checkered columns). Cell growth was evaluated by measuring the change in OD₆₀₀ after 20 hours. Cell growth in media without additives is denoted as Control, shown with a blank negative control and a negative growth control using 50 μg/mL chloramphenicol (solid columns).

FIG. 15 shows results of an in vitro linear PEI assay against MRSA. LPEI alone does not affect the growth of MRSA through 53 μg/mL (striped columns); addition of 9.3 μg/mL ampicillin provides some synergistic effect, though not enough to inhibit MRSA growth (thatched columns). Growth of MRSA was evaluated by measuring the change in OD₆₀₀ after 20 hours. Cell growth in media (Control) or media with 1% DMSO show the increase in OD₆₀₀ (solid columns), while 50 μg/mL chloramphenicol serves as a negative control.

FIG. 16 shows microphotographs of sections having BPEI binding to MRSA and E. coli. MRSA and E. coli were treated with either BPEI-conjugated AlexaFluor 488 (top row), only Alexa Fluor 488 (middle row), or only BPEI (bottom row). The left column of images shows the net fluorescence intensity for each channel. The middle columns shows fluorescence intensity of cells stained with DAPI. The right column shows the merged images of both AlexaFluor 488 and DAPI staining, where the fluorescence intensities within each image were normalized relative to the intensity of fluorescence of MRSA treated with BPEI conjugated AlexaFluor 488. Scale bar=5 μm.

FIG. 17A-B shows ³¹P NMR spectra of WTA before (A) and after (B) the addition of low-Mw BPEI demonstrating significant changes in phosphate chemical shift caused by changes in the chemical environment. The P-31 signals near 4 ppm are correlated with the proton signals of NAG sugar groups. However, clear identification of specific interactions is prevented by the heterogeneous nature of WTA functional groups, BPEI branching, and WTA:BPEI binding interactions.

FIG. 17C-D shows ¹H/³¹P heteronuclear multiple-bond correlation (HMBC) NMR spectra of WTA before (C) and after (D) the addition of low-Mw BPEI demonstrating significant changes in phosphate chemical shift caused by changes in the chemical environment.

FIG. 18 shows an in vitro checkerboard assay of the effectiveness of BPEI in restoring methicillin efficacy against MRSA. An optical density (OD₆₀₀)<0.05 was used as an indicator of growth inhibition. Synergy was confirmed using FIC values.

FIG. 19 shows an in vitro checkerboard assay of the effectiveness of BPEI in restoring ampicillin efficacy against MRSA. An optical density (OD₆₀₀)<0.05 was used as an indicator of growth inhibition. Synergy was confirmed using FIC values.

FIG. 20 is an in vitro checkerboard assay showing the effectiveness of BPEI in restoring ampicillin efficacy against MRSA. An optical density (OD₆₀₀)<0.05 was used as an indicator of growth inhibition. Synergy was confirmed using FIC values.

FIG. 21 is an in vitro checkerboard assay showing that Vancomycin (D) has an MIC of 4 μg/ml, denoted as intermediate resistance, but does not exhibit synergy with BPEI. Instead, the effect is additive.

FIG. 22 shows that no potentiation of the effect of ampicillin on MRSA occurs when the linear polymer (LPEI) is used in combination with ampicillin.

### DETAILED DESCRIPTION

In at least certain embodiments, the present disclosure is directed to novel compositions comprising antibiotics against which certain bacteria (e.g., Methicillin-resistant Staphylococcus aureus (MRSA)) strains have previously been resistant. In other words, the bacterial strains have become resensitized to these novel antibiotic formulations which comprise historical antibiotics, such as, but not limited to, the β-lactams, for example, methicillin, amoxicillin, and ampicillin, and others described elsewhere herein. In particular, results provided herein show that the lost anti-MRSA effectiveness of certain FDA-approved antibiotics, such as ampicillin (or other antibiotic listed elsewhere herein), can be restored via a synergistic effect when they are administered in the presence of branched poly(ethylenimine) (BPEI), a cationic polyamine. Further, the effective levels (i.e., the minimum inhibitory concentration (MIC)) of certain other antibiotics can be substantially reduced (e.g., by about ten-fold) when administered with BPEI.

Before further describing various embodiments of the compositions and methods of the present disclosure in more detail by way of exemplary description, examples, and results, it is to be understood that the present disclosure is not limited in application to the details of methods and compositions as set forth in the following description. The description provided herein is intended for purposes of illustration only and is not intended to be construed in a limiting sense. The inventive concepts of the present disclosure are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the present disclosure may be practiced without these specific details. In other instances, features which are well known to persons of ordinary skill in the art have not been described in detail to avoid unnecessary complication of the description. It is intended that all alternatives, substitutions, modifications and equivalents apparent to those having ordinary skill in the art are included within the scope of the present disclosure as defined herein. Thus, while the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB.

Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately”, where used herein when referring to a measurable value such as an amount, a temporal duration, and the like, are meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

The active agents of the combination therapies of the present disclosure may be used or administered conjointly. As used herein the terms “conjointly” or “conjoint administration” refers to any form of administration of two or more different biologically-active compounds (i.e., active agents) such that the second compound is administered while the previously administered therapeutic compound is still effective in the body, whereby the two or more compounds are simultaneously active in the patient, enabling a synergistic interaction of the compounds. For example, the different therapeutic compounds can be administered either in the same formulation, or in separate formulations, either concomitantly (together) or sequentially. When administered sequentially the different compounds may be administered immediately in succession, or separated by a suitable duration of time, as long as the active agents function together in a synergistic manner. In certain embodiments, the different therapeutic compounds can be administered within one hour of each other, within two hours of each other, within 3 hours of each other, within 6 hours of each other, within 12 hours of each other, within 24 hours of each other, within 36 hours of each other, within 48 hours of each other, within 72 hours of each other, or more. Thus an individual who receives such treatment can benefit from a combined effect of the different therapeutic compounds. In one example of conjoint administration, a β-lactam antibiotic and a potentiating compound (e.g., a BPEI) are administered to the surface in sequential or simultaneous steps, or as a composition comprising both the β-lactam antibiotic and the potentiating compound. The term BPEI, where used herein, is also intended to refer to pegylated BPEI (PEG-BPEI) molecules, unless expressly indicated otherwise.

The term “pharmaceutically acceptable” refers to compounds and compositions which are suitable for administration to humans and/or animals without undue adverse side effects such as toxicity, irritation and/or allergic response commensurate with a reasonable benefit/risk ratio.

By “biologically active” is meant the ability to modify the physiological system of an organism without reference to how the active agent has its physiological effects.

As used herein, “pure,” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The terms “subject” and “patient” are used interchangeably herein and will be understood to refer to a warm blooded animal, particularly a mammal, and more particularly, humans. Animals which fall within the scope of the term “subject” as used herein include, but are not limited to, dogs, cats, rats, mice, guinea pigs, chinchillas, horses, goats, ruminants such as cattle, sheep, swine, poultry such as chickens, geese, ducks, and turkeys, zoo animals, Old and New World monkeys, and non-human primates. Veterinary diseases and conditions which may be treated with the compositions of the present disclosure include, but are not limited to, anthrax, listeriosis, leptospirosis, clostridial and corynebacterial infections, streptococcal mastitis, and keratoconjunctivitis in ruminants; erysipelas, streptococcal and clostridial infections in swine; tetanus, strangles, streptococcal and clostridial infections, and foal pneumonia in horses; urinary tract infections, and streptococcal and clostridial infections in dogs and cats; and necrotic enteritis, ulcerative enteritis and intestinal spirochetosis in poultry.

“Treatment” refers to therapeutic treatments. “Prevention” refers to prophylactic or preventative treatment measures. The term “treating” refers to administering the composition to a patient for therapeutic purposes.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “β-lactam antibiotic” refers to the class of antibiotic agents that have a β-lactam ring or derivatized β-lactam ring in their molecular structures. Examples of such β-lactam antibiotics include but are not limited to, penams, including but not limited to, penicillin, benzathine penicillin, penicillin G, penicillin V, procaine penicillin, ampicillin, amoxicillin, methicillin, cloxacillin, dicloxacillin, flucloxacillin, nafcillin, oxacillin, temocillin, mecillinam, carbenicillin, ticarcillin, and azlocillin, mezlocillin, piperacillin; cephems, including but not limited to, cephalosporin C, cefoxitin, cephalosporin, cephamycin, cephem, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline; carbapenems and penems including but not limited to, biapenem, doripenem, ertapenem, earopenem, imipenem, primaxin, meropenem, panipenem, razupenem, tebipenem, and thienamycin; and monobactams including but not limited to, aztreonam, tigemonam, nocardicin A, and tabtoxinine β-lactam.

The terms “effective amount”, “antibacterially-effective amount”, or “therapeutically-effective amount” refers to an amount of an antibiotic composition (β-lactam antibiotic plus a BPEI) which is sufficient to exhibit a detectable therapeutic effect against bacterial growth without excessive adverse side effects (such as toxicity, irritation and allergic response) commensurate with a reasonable benefit/risk ratio when used in the manner as described herein. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance for a given subject or patient. However, the effective amount for a given situation can be determined by one of ordinary skill in the art using routine experimentation based on the information provided herein.

In some embodiments of the present disclosure a pegylated or non-pegylated low molecular weight (“low Mw”) BPEI is used in combination with an anti-bacterial agent to treat and/or inhibit a resistant bacterial infection and/or the growth of resistant bacterial infection, e.g., by sensitizing a bacterium that was previously resistant or substantially resistant to an antibacterial agent, are described herein. In certain non-limiting embodiments the low Mw BPEI of the present disclosure has a Mw in range of, for example, 0.1 kDa (kilodaltons) to 25 kDa. Examples of BPEI compounds which may be used in various embodiments of the present disclosure include but are not limited to those shown in U.S. Pat. Nos. 7,238,451 and 9,238,716, and U.S. Published application 2014/0369953, the entireties of which are hereby incorporated by reference herein.

Minimum inhibitory concentration (MIC) of an antibiotic for a particular bacterial strain is defined as the lowest concentration of the antibiotic that is required to inhibit the growth of the bacterium. The MIC is determined by finding the concentration of antibiotic at which there is no growth of the bacterium.

Sensitizing, or sensitization, as the term is used herein, is the process of lowering the MIC of an antibiotic for a resistant bacterial strain to a value that is below the resistance breakpoint for the bacterial strain, thereby causing the bacterium to be more susceptible to that antibiotic.

A breakpoint (or resistance breakpoint) is defined as a concentration of an antibiotic (mg/L) which defines whether a strain of bacteria is susceptible or resistant to the antibiotic. If the MIC is less than or equal to the breakpoint, the strain is considered susceptible to the antibiotic. If the MIC is greater than the breakpoint, the strain is considered intermediate or resistant to the antibiotic.

The compounds and compositions of the present disclosure can be used to treat a subject having resistant bacterial infection, e.g., by administering BPEI in combination with an antibiotic. The combinations of BPEI and the antibacterial agent can result in sensitization of a resistant bacterial strain, e.g., the resistant bacterial strain has a reduced MIC of either the BPEI, or the antibacterial agent, or both, so that the MIC is below the resistance breakpoint for the bacterial strain.

As used herein “resistant bacterial strain” means a bacterial strain which is resistant to an antibacterial agent, e.g. having an MIC that is greater than the resistance breakpoint (as the term is defined herein). In certain embodiments the MIC of a resistant bacterial strain will be at least 2-, 4-, 8-, 10-, 16-, 32-, 64-, or 100-fold greater than for that seen with a non-resistant bacterial strain for a selected antibacterial agent. As used herein, rendering or transforming a resistant bacterial into a sensitive bacterial strain means reducing the MIC, e.g., by at least 2-, 4-, 8-, 10-, 16-, 32-, 64-, or 100-fold.

The term “biofilm” as used herein refers to an aggregate of microorganisms in which cells adhere to each other and/or to a surface. These adherent cells are frequently embedded within a self-produced matrix of extracellular polymeric substance. The microorganisms comprising a biofilm may include bacteria, archaea, fungi, protozoa, algae, or combinations thereof. In particular embodiments, the biofilm comprises a bacterium (such as described elsewhere herein) such that the biofilm is a bacterial biofilm.

In some embodiments, the surface having the biofilm thereon may be a surface of a medical device. In some embodiments, the biofilm may be partially or entirely implantable in a body of a subject. For example, the medical device may be a catheter. Non-limiting examples of suitable catheters include intravascular catheters (such as, e.g., arterial catheters, central venous catheters, hemodialysis catheters, peripheral and venous catheters), endovascular catheter microcoils, peritoneal dialysis catheters, urethral catheters, catheter access ports, shunts, intubating and tracheotomy tubes. For example, the medical device may be a peripherally inserted central catheter (PICC) line. In another embodiment, the implantable device may be a cardiac device. Examples of cardiac devices include, but are not limited to, cardiac stents, defibrillators, heart valves, heart ventricular assist devices, OEM component devices, pacemakers, and pacemaker wire leads. In further embodiments, the medical device may be an orthopedic device. Non-limiting examples of suitable orthopedic devices include implants such as knee replacements, hip replacements, shoulder replacements, other joint replacements and prostheses, spinal disc replacements, orthopedic pins, plates, screws, rods, and orthopedic OEM components. In other embodiments, the medical device may include endotracheal tubes, nasogastric feeding tubes, gastric feeding tubes, synthetic bone grafts, bone cement, biosynthetic substitute skin, vascular grafts, surgical hernia mesh, embolic filter, ureter renal biliary stents, urethral slings, gastric bypass balloons, gastric pacemakers, insulin pumps, neurostimulators, penile implants, soft tissue silicone implants, intrauterine contraceptive devices, cochlear implants, dental implants and prosthetics, voice restoration devices, ophthalmic devices such as contact lenses.

In some embodiments, the surface having the biofilm thereon is a surface or within the body of a subject. For example, the subject may be a veterinary subject. Non-limiting examples of suitable veterinary subjects include companion animals such as cats, dogs, rabbits, horses, and rodents such as gerbils; agricultural animals such as cows, cattle, pigs, goats, sheep, horses, deer, chickens and other fowl; zoo animals such as primates, elephants, zebras, large cats, bears, and the like; and research animals such as rabbits, sheep, pigs, dogs, primates, chinchillas, guinea pigs, mice, rats and other rodents. For instance, the composition may be used to treat skin infections, soft tissue infections, and/or mastitis in veterinary subjects such as companion animals and/or agricultural animals. The veterinary subject may be suffering from or diagnosed with a condition needing treatment, or the veterinary subject may be treated prophylactically.

In other embodiments, the subject having the surface having the biofilm thereon may be a human health care patient. Non-limiting examples of suitable health care patients include ambulatory patients, surgery patients, medical implantation patients, hospitalized patients, long-term care patients, and nursing home patients. In still other embodiments, the subject may be a health care worker. Suitable health care workers include those with direct and indirect access to patients, medical equipment, and medical facilities.

In some embodiments the combination of the BPEI and the antibiotic results in a reduction in the MIC of the BPEI and/or the antibiotic of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or at least about 99.5%.

The antimicrobial (antibacterial) action of two or more active agents is considered additive if the combined action merely results from the addition of the effects the individual components would have in isolation. In contrast, the antimicrobial action of two or more active compounds is considered to be synergistic if the combined effect of the two or more compounds is stronger than expected based on the assumption of additivity.

More particularly, the term “synergy” or “synergistic” as used herein, refers to an outcome when two agents (e.g., BPEI and an antibiotic) are used in combination, wherein the combination of the agents acts so as to require a smaller amount of each individual agent than would be required of that agent to be efficacious in the absence of the other agent, for example, with lower dosages of the first agent than would be required in the absence of the second agent. In some embodiments, use of synergistic agents can result in the beneficial effect of less overall use of an agent. Typically, evidence of synergistic antimicrobial action may be provided at concentrations below the MICs of each of the components when taken individually. However, a synergistic interaction can also occur when the concentration of one or more of the active compounds is raised above its MIC (when taken individually).

The fractional inhibitory concentration (FIC) as used herein is a measure of the interaction of two agents, such as an antibiotic and a BPEI compound, used together, and is an indicator of synergy. FIC uses a value of the MIC of each of the independent agents, e.g., MIC_{A} and MIC_{B} for agents A and B, for a particular bacterium as the basis, then takes the concentration of each component in a mixture where an MIC_{(A in B)} is observed. For example, for a two component system of agents A and B, MIC_{(A in B)} is the concentration of A in the compound mixture and MIC_{(B in A)} is the concentration of B in the compound mixture. The FIC is defined as follows:

FIC_{A}=(MIC_{(A in B)}/MIC_{A}) Eqn. 1

FIC_{B}=(MIC_{(B in A)}/MIC_{B}) Eqn. 2

FIC_{A+B}=FIC_{A}+FIC_{B} Eqn. 3

Synergism (i.e., the two compounds together provide a synergistic effect) is defined herein as occurring when FIC_{A+B}≤0.5. The mixture is defined as having an additive effect when 1≤FIC_{A+B}≤4. When, FIC_{A+B}>4 the mixture is considered to have an antagonistic interaction. An example of how FIC is used to determine synergism is shown in U.S. Pat. No. 8,338,476, the entirety of which is incorporated herein by reference in its entirety.

In certain embodiments of the present disclosure, the BPEI/antibiotic combination results in an FIC less than about 0.55, or less than about 0.5, or less than about 0.4, or less than about 0.3, or less than about 0.2, or less than about 0.1, or less than about 0.05, or less than about 0.02, or less than about 0.01, or less than about 0.005, or less than about 0.001. In some embodiments, the combination results in a bactericidal activity at least about 2, at least about 2.5, at least about 3, at least about 3.5, at least about 4, at least about 4.5, or at least about 5 logs more effective than the most effective individual activity, e.g., the activity of the BPEI or the antibiotic agent.

As used herein, “resistant microorganism or bacterium” means an organism which has become resistant to an anti-bacterial agent. In certain embodiments an MIC of a resistant bacterium will be at least, 2-, 4-, 8-, 10-, 16-, 32-, 64-, or 100-fold greater than that seen with a non-resistant bacterium for a particular anti-bacterial agent. As used herein, the term “resistance breakpoint” is the threshold concentration of an antibacterial agent above which a bacterium is considered resistant, as defined above.

In certain non-limiting embodiments, the antibiotic/BPEI composition is formulated to contain a mass ratio in a range of 100:1 (e.g., 100 mg antibiotic per 1 mg of BPEI additive), to 1:1 (1 mg antibiotic per 1 mg BPEI), to 1:100 (1 mg antibiotic per 100 mg BPEI), or more particularly, a mass ratio in a range of 75:1 (e.g., 75 mg antibiotic per 1 mg of BPEI additive), to 1:1 (1 mg antibiotic per 1 mg BPEI), to 1:75 (1 mg antibiotic per 75 mg BPEI), or more particularly, a mass ratio in a range of 64:1 (e.g., 64 mg antibiotic per 1 mg of BPEI additive), to 1:1 (1 mg antibiotic per 1 mg BPEI), to 1:64 (1 mg antibiotic per 64 mg BPEI), or more particularly, a mass ratio in a range of 50:1 (e.g., 50 mg antibiotic per 1 mg of BPEI additive), to 1:1 (1 mg antibiotic per 1 mg BPEI), to 1:50 (1 mg antibiotic per 50 mg BPEI), or more particularly, a mass ratio in a range of 32:1 (e.g., 32 mg antibiotic per 1 mg of BPEI additive), to 1:1 (1 mg antibiotic per 1 mg BPEI), to 1:32 (1 mg antibiotic per 32 mg BPEI), or more particularly, a mass ratio in a range of 24:1 (e.g., 24 mg antibiotic per 1 mg of BPEI additive), to 1:1 (1 mg antibiotic per 1 mg BPEI), to 1:24 (1 mg antibiotic per 24 mg BPEI), or more particularly, a mass ratio in a range of 16:1 (e.g., 16 mg antibiotic per 1 mg of BPEI additive), to 1:1 (1 mg antibiotic per 1 mg BPEI), to 1:16 (1 mg antibiotic per 16 mg BPEI), or more particularly, a mass ratio in a range of 10:1 (e.g., 10 mg antibiotic per 1 mg of BPEI additive), to 1:1 (1 mg antibiotic per 1 mg BPEI), to 1:10 (1 mg antibiotic per 10 mg BPEI), or more particularly, a mass ratio in a range of 8:1 (e.g., 8 mg antibiotic per 1 mg of BPEI additive), to 1:1 (1 mg antibiotic per 1 mg BPEI), to 1:8 (1 mg antibiotic per 8 mg BPEI), or more particularly, a mass ratio in a range of 4:1 (e.g., 4 mg antibiotic per 1 mg of BPEI additive), to 1:1 (1 mg antibiotic per 1 mg BPEI), to 1:4 (1 mg antibiotic per 4 mg BPEI), or any range comprising a combination of said ratio endpoints, such as for example, a mass ratio in a range of 64:1 (e.g., 64 mg antibiotic per 1 mg of BPEI additive), to 1:4 (1 mg antibiotic per 4 mg BPEI), or a mass ratio in a range of 32:1 (e.g., 32 mg antibiotic per 1 mg of BPEI additive), to 1:16 (1 mg antibiotic per 16 mg BPEI).

In certain non-limiting embodiments, the dosage of the antibiotic/BPEI composition administered to a subject could be in a range of 1 μg per kg of subject body mass to 1000 mg/kg, or in a range of 5 μg per kg to 500 mg/kg, or in a range of 10 μg per kg to 300 mg/kg, or in a range of 25 μg per kg to 250 mg/kg, or in a range of 50 μg per kg to 250 mg/kg, or in a range of 75 μg per kg to 250 mg/kg, or in a range of 100 μg per kg to 250 mg/kg, or in a range of 200 μg per kg to 250 mg/kg, or in a range of 300 μg per kg to 250 mg/kg, or in a range of 400 μg per kg to 250 mg/kg, or in a range of 500 μg per kg to 250 mg/kg, or in a range of 600 μg per kg to 250 mg/kg, or in a range of 700 μg per kg to 250 mg/kg, or in a range of 800 μg per kg to 250 mg/kg, or in a range of 900 μg per kg to 250 mg/kg, or in a range of 1 mg per kg to 200 mg/kg, or in a range of 1 mg per kg to 150 mg/kg, or in a range of 2 mg per kg to 100 mg/kg, or in a range of 5 mg per kg to 100 mg/kg, or in a range of 10 mg compound per kg to 100 mg/kg, or in a range of 25 mg per kg to 75 mg/kg. For example, in certain non-limiting embodiments, the composition could contain antibiotic in a range of 0.1 mg/kg to 10 mg/kg, and BPEI in a range of 0.1 mg/kg to 10 mg/kg, or any range comprising a combination of said ratio endpoints, such as, for example, a range of 10 μg/kg to 10 mg/kg of the antibiotic/BPEI composition. In some embodiments, the antibiotic and/or potentiating compound is administered at a dose of about 0.1 mg/kg to about 50 mg/kg. In particular embodiments, the subject is a pediatric patient, which means under 18 years of age for a human patient. For a pediatric patient, in some embodiments the antibiotic and/or potentiating compound is administered about 10 mg/kg to about 50 mg/kg intravenously or intramuscularly every 6 to 12 hours or about 12.5 mg/kg orally every 6 hours.

The BPEI used in the present formulations may have a molecular weight in a range of, for example, from 0.1 kDa (kilodaltons), to 0.25 kDa, to 0.50 kDa, to 0.75 kDa, to 1.0 kDa, to 2 kDa, to 3 kDa, to 4 kDa, to 5 kDa, to 6 kDa, to 7 kDa, to 8 kDa, to 9 kDa, to 10 kDa, to 12.5 kDa, to 15 kDa, to 17.5 kDa, to 20 kDa, to 22.5 kDa, to 25 kDa, including any fractional or integeric value within said range. Also, the percentage of primary amine—to—secondary amine—to—tertiary amine in the BPEI can be varied. For example, the BPEI may have a higher primary amine content as compared to the secondary amine and/or tertiary amine content.

The PEG molecules used in the pegylated BPEI formulations of the present disclosure may have an average molecular weight (MW) in a range of, for example, from 0.1 kDa (kilodaltons), to 0.2 kDa, to 0.3 kDa, to 0.4 kDa, to 0.50 kDa, to 0.6 kDa, to 0.7 kDa, to 0.8 kDa, to 0.9 kDa, to 1.0 kDa, to 1.1 kDa, to 1.2 kDa, to 1.3 kDa, to 1.4 kDa, to 1.5 kDa, to 1.6 kDa, to 1.7 kDa, to 1.8 kDa, to 1.9 kDa, to 2 kDa, to 2.1 kDa, to 2.2 kDa, to 2.3 kDa, to 2.4 kDa, to 2.5 kDa, to 2.6 kDa, to 2.7 kDa, to 2.8 kDa, to 2.9 kDa, to 3 kDa, to 3.1 kDa, 3.2 kDa, to 3.3 kDa, to 3.4 kDa, to 3.5 kDa, to 3.6 kDa, to 3.7 kDa, to 3.8 kDa, to 3.9 kDa, to 4 kDa, to 4.1 kDa, 4.2 kDa, to 4.3 kDa, to 4.4 kDa, to 4.50 kDa, to 4.6 kDa, to 4.7 kDa, to 4.8 kDa, to 4.9 kDa, to 5 kDa, to 5.5 kDa, to 6 kDa, to 6.5 kDa, to 7 kDa, to 7.5 kDa, to 8 kDa, to 9 kDa, to 10 kDa, including any fractional or integeric value within said range, such as 150 Da to 2500 Da (i.e., 0.15 kDa to 2.5 kDa), 200 Da to 1750 Da (i.e., 0.2 kDa to 1.75 kDa), 250 Da to 1500 Da (i.e., 0.25 kDa to 1.5 kDa), and 300 Da to 1250 Da (i.e., 0.3 kDa to 1.25 kDa).

In a non-limiting embodiment, PEGylation of a low MW BPEI (e.g., 600-Da BPEI) can be performed with mPEG-Epoxide (e.g., 350 MW, 550 MW, 750 MW, 1000 MW, or 2000 MW). The reaction proceeds in anhydrous ethanol at 60° C., where the epoxide-ring signals disappear. In non-limiting embodiments, the BPEI may be decorated with the 350, 550, 750, 1000, or 2000 MW PEG separately in three different molar ratios of 1:1, 2:1, and 3:1 each.

Poly(ethylenimine)s can be readily modified by covalently attaching methyl end-capped polyethylene glycol chains (PEGs, CH₃[OCH₂CH₂]ₙ— with various n values) to its amine nitrogens. This can be accomplished by various means including nucleophilic substitution by the nitrogens of poly(ethylenimines) on PEGs with terminal leaving groups (such as halogens or sulfonates), by reductive amination on PEGs with carboxylic acid end-groups, and by conjugate addition of the nitrogens of poly(ethylenimines) to PEGs with acrylate end-groups. However, perhaps the most convenient strategy is to react the nitrogens of poly(ethylenimines) with PEGs having glycidyl epoxide end-groups to form β-aminoalcohol linkages. PEG-type diepoxides react with ferrocenyl-modified poly(ethylenimine)s, even in aqueous media. It is generally reported that 1° amines are more reactive with glycidyl epoxides than 2° amines, and 3° amines are essentially non-reactive as they do not have protons to transfer. However, the reported selectivity's are modest, ranging from 1°/2° reactivity ratios of about 1.5/1 to about 16/1. Therefore, “PEGylation” of BPEIs will occur mostly on the 1° amines and reaction temperatures below 80° C. can be used to ensure 1° amines are the predominant reaction site. PEG-BPEI compositions having an approximate 1:1 ratio retains efficacy (Table 3) and has a higher maximum tolerable dose than 600-Da BPEI itself. The reaction can be performed in absolute ethanol and can be conveniently followed using ¹H-NMR spectroscopy by observing the characteristic epoxide signals disappear. We can also draw on the other amine reactive PEGs, such as mPEG-Mesylate and mPEG-Tosylate.

In one non-limiting embodiment, an exemplary library of cationic potentiators based on 600-Da BPEI with capped amines can be created. In general, 600-Da BPEI is less toxic than 1200, 1800 or 10,000-Da BPEI, reducing the number of primary amines further reduces toxicity. In one embodiment epoxide ring-opening chemistry can be used to react the primary amines of 600-Da BPEI with moieties having glycidyl epoxide end-groups to form β-amino alcohol linkages. Primary amines of 600-Da BPEI were reacted with an ethyl, diglyme, or PEG molecules having a glycidyl epoxide end-group.

The single-step reaction occurs under mild conditions (ethanol solvent at °60 C) with minimal workup (single pass through a silica gel column). The different potentiators balance cationic properties (for binding to the anionic targets) and reducing the number of amines with hydrophilic groups (to reduce toxicity). These criteria are met by capping amines with ethyl, diglyme, and PEG groups. Unlike rigid cyclic peptides, capped BPEIs are flexible structures that can access anionic sites on the flexible LPS and WTA molecules.

In addition to the PEGylation and epoxide reactions, the compositions and methods of the present disclosure can utilize BPEI molecules which have been modified to form other compounds, such as but not limited to anhydrides, acrylamides, acrylates, methacrylates, methacrylamides, and bis-methacrylates and bis-acrylamides to form bridged dimers, e.g., (BPEI)-linker-(BPEI).

The antibiotic and BPEI can be administered together in a single formulation (dose), or together (simultaneously) in separate formulations (doses), or sequentially, whereby administration of the antibiotic dosage is followed by the BPEI dosage, or administration of the BPEI dosage is followed by administration of the antibiotic dosage. The dosage(s) can be administered, for example but not by way of limitation, on a one-time basis, or administered at multiple times (for example but not by way of limitation, from one to five times per day, or once or twice per week), or continuously via a venous drip, depending on the desired therapeutic effect. In one non-limiting example of a therapeutic method of the presently disclosed inventive concepts, the composition is provided in an IV infusion. Administration of the compounds used in the pharmaceutical composition or to practice the method of the presently disclosed inventive concepts can be carried out in a variety of conventional ways, such as, but not limited to, orally, by inhalation, rectally, or by cutaneous, subcutaneous, intraperitoneal, vaginal, or intravenous injection. Oral formulations may be formulated such that the compounds pass through a portion of the digestive system before being released, for example it may not be released until reaching the small intestine, or the colon.

In some embodiments the antibiotic and the potentiator compound are in the same composition. In other embodiments the antibiotic and the potentiator compound are administered simultaneously in the same or different compositions. A subject is administered an antibiotic up to 24 hours prior to administration of the potentiator compound in some cases. In others, the potentiator compound is administered up to 24 hours prior to administration of the antibiotic. In some embodiments, the antibiotic and potentiator compound are administered within 0.5, 1, 2, 3, 4, 6, 8, 10, 12, 14, 16, 18, 20, 22, or 24 hours of each other.

As noted above, in certain embodiments, the compositions of the present disclosures may be applied topically to an external or internal wound to treat a planktonic or biofilm bacterial infection in or on the wound. The treated wounds may be acute wounds, such as abrasions, lacerations, punctures, avulsions and incisions, or chronic wounds, or “non-healing” wounds such as diabetic foot ulcers, venous leg ulcers, pressure ulcers (e.g., bed sores), wounds due to arterial insufficiency, radiation wounds, and non-healing surgical wounds (e.g., due to abdominal surgery).

The composition for topical or internal application may be provided in any suitable solid, semi-solid, or liquid form. In certain embodiments, the topical composition may be provided in or be disposed in a carrier(s) or vehicle(s) such as, for example, creams, pastes, gums, lotions, gels, foams, ointments, emulsions, suspensions, aqueous solutions, powders, lyophilized powders, solutions, granules, foams, drops, eye drops, adhesives, sutures, aerosols, sprays, sticks, soaps, bars of soap, balms, body washes, rinses, tinctures, gel beads, gauzes, wound dressings, bandages, cloths, towelettes, stents, and sponges. Non-limiting examples of formulations of such carriers and vehicles include, but are not limited to, those shown in “Remington, The Science and Practice of Pharmacy, 22^{nd} ed., 2012, edited by Loyd V. Allen, Jr”.

Creams are emulsions of water in oil (w/o), or oil in water (o/w). 0/w creams spread easily and do not leave the skin greasy and sticky. W/o creams tend to be more greasy and more emollient. Ointments are semi-solid preparations of hydrocarbons and the strong emollient effect makes it useful in cases of dry skin. The occlusive effect enhances penetration of the active agent and improves efficacy. Pastes are mixtures of powder and ointment. The addition of the powder improves porosity thus breathability. The addition of the powder to the ointment also increases consistency so the preparation is more difficult to rub off or contact non-affected areas of the skin. Lotions are liquid preparations in which inert or active medications are suspended or dissolved. For example, an o/w emulsion with a high water content gives the preparation a liquid consistency of a lotion. Most lotions are aqueous of hydroalcoholic systems wherein small amounts of alcohol are added to aid in solubilization of the active agent and to hasten evaporation of the solvent from the skin surface. Gels are transparent preparations containing cellulose ethers or carbomer in water, or a water-alcohol mixture. Gels liquefy on contact with the skin, dry, and leave a thin film of active medication.

A person with ordinary skill in the art will be capable of determining the effective amount of the composition needed for a particular treatment. Such amount may depend on the strength of the composition or extent of the wound to be treated. Although a person with ordinary skill in the art will know how to select a treatment regimen for a specific condition. In a non-limiting example, a dosage of the composition comprising about 0.01 mg to about 1000 mg of the active agent (antibiotic plus a BPEI) per ml may be applied 1 to 2 to 3 to 4 to 5 to 6 times per day or more to the affected area. It is foreseeable in some embodiments that the composition is administered over a period of time. The composition may be applied for a day, multiple days, a week, multiple weeks, a month, or even multiple months in certain circumstances. Alternatively, the composition may be applied only once when the skin condition is mild.

In certain embodiments, the composition may comprise the active agents in a concentration of, but is not limited to, 0.0001 M to 1 M, for example, or 0.001 M to 0.1 M. The composition may comprise about 0.01 to about 1000 milligrams of the active agents per ml of carrier or vehicle with which the active agents are combined in a composition or mixture. The composition may comprise about 1 wt % to about 90 wt % (or 1 mass % to about 90 mass %) of one or more shikimate analogues and about 10 wt % to about 99 wt % (or 10 mass % to about 99 mass %) of one or more secondary compounds (where “wt %” is defined as the percentage by weight of a particular compound in a solid or liquid composition, and “mass %” is defined as the percentage by mass of a particular compound in a solid or liquid composition).

The topical compositions may further comprise ingredients such as propylene glycol, sodium stearate, glycerin, a surfactant (e.g., sodium laurate, sodium laureth sulfate, and/or sodium lauryl sulfate), and water, and optionally, sorbitol, sodium chloride, stearic acid, lauric acid, aloe vera leaf extract, pentasodium penetrate, and/or tetrasodium etidronate.

The topical compositions may be formulated with liquid or solid emollients, solvents, thickeners, or humectants. Emollients include, but are not limited to, stearyl alcohol, mink oil, cetyl alcohol, oleyl alcohol, isopropyl laurate, polyethylene glycol, olive oil, petroleum jelly, palmitic acid, oleic acid, and myristyl myristate. Emollients may also include natural butters extracted from various plants, trees, roots, or seeds. Examples of such butters include, but are not limited to, shea butter, cocoa butter, avocado butter, aloe butter, coffee butter, mango butter, or combination thereof.

Suitable materials which may be used in the compositions as carriers or vehicles or secondary compounds or solvents include, but are not limited to, propylene glycol, ethyl alcohol, isopropanol, acetone, diethylene glycol, ethylene glycol, dimethyl sulfoxide, and dimethyl formamide. Suitable humectants include, but are not limited to, acetyl arginine, algae extract, Aloe barbadensis leaf extract, 2,3-butanediol, chitosan lauroyl glycinate, diglycereth-7 malate, diglycerin, diglycol guanidine succinate, erythritol, fructose, glucose, glycerin, honey, hydrolyzed wheat protein/polyethylene glycol-20 acetate copolymer, hydroxypropyltrimonium hyaluronate, inositol, lactitol, maltitol, maltose, mannitol, mannose, methoxypolyethylene glycol, myristamidobutyl guanidine acetate, polyglyceryl sorbitol, potassium pyrollidone carboxylic acid (PCA), propylene glycol (PGA), sodium pyrollidone carboxylic acid (PCA), sorbitol, and sucrose. Other humectants may be used for yet additional embodiments of the compositions of the present disclosure.

Suitable thickeners include, but are not limited to, polysaccharides, in particular xantham gum, guar-guar, agar-agar, alginates, carboxymethylcellulose, relatively high molecular weight polyethylene glycol mono- and diesters of fatty acids, polyacrylates, polyvinyl alcohol and polyvinylpyrrolidone, surfactants such as, for example, ethoxylated fatty acid glycerides, esters of fatty acids with polyols such as, for example, pentaerythritol or trimethylpropane, fatty alcohol ethoxylates or alkyl oligoglucosides, and electrolytes, such as sodium chloride and ammonium chloride.

The topical compositions may further comprise one or more penetrants, compounds facilitating penetration of active ingredients into the skin of a patient. Non-limiting examples of suitable penetrants include isopropanol, polyoxyethylene ethers, terpenes, cis-fatty acids (oleic acid, palmitoleic acid), acetone, laurocapram dimethyl sulfoxide, 2-pyrrolidone, oleyl alcohol, glyceryl-3-stearate, cholesterol, myristic acid isopropyl ester, and propylene glycol. Additionally, the compositions may include surfactants or emulsifiers for forming emulsions. Either a water-in-oil or oil-in-water emulsion may be formulated. Examples of suitable emulsifiers include, but are not limited to, stearic acid, cetyl alcohol, PEG-100, stearate and glyceryl stearate, cetearyl glucoside, polysorbate 20, methylcellulose, sodium carboxymethylcellulose, glycerin, bentonite, ceteareth-20, cetyl alcohol, cetearyl alcohol, lanolin alcohol, riconyl alcohol, self-emulsifying wax (e.g., Lipowax P), cetyl palmitate, stearyl alcohol, lecithin, hydrogenated lecithin, steareth-2, steareth-20, and polyglyceryl-2 stearate.

In some formulations, such as in aerosol form, the composition may also include a propellant. For example, hydrofluoroalkanes (HFA) such as either HFA 134a (1,1,1,2-tetrafluoroethane) or HFA 227 (1,1,1,2,3,3,3-heptafluoropropane) or combinations of the two, may be used since they are widely used in medical applications. Other suitable propellants include, but are not limited to, mixtures of volatile hydrocarbons, typically propane, n-butane and isobutane, dimethyl ether (DME), methylethyl ether, nitrous oxide, and carbon dioxide. Those skilled in the art will readily appreciate that emollients, solvents, thickeners, humectants, penetrants, surfactants or emulsifiers, and propellants, other than those listed may also be employed.

When a therapeutically effective amount of the composition(s) is administered orally, it may be in the form of a solid or liquid preparations such as capsules, pills, tablets, lozenges, melts, powders, suspensions, solutions, elixirs or emulsions. Solid unit dosage forms can be capsules of the ordinary gelatin type containing, for example, surfactants, lubricants, and inert fillers such as lactose, sucrose, and cornstarch, or the dosage forms can be sustained release preparations. The pharmaceutical composition(s) may contain a solid carrier, such as a gelatin or an adjuvant. The tablet, capsule, and powder may contain from about 0.05 to about 95% of the active substance compound by dry weight. When administered in liquid form, a liquid carrier such as water, petroleum, oils of animal or plant origin such as peanut oil, mineral oil, soybean oil, or sesame oil, or synthetic oils may be added. The liquid form of the pharmaceutical composition(s) may further contain physiological saline solution, dextrose or other saccharide solution, or glycols such as ethylene glycol, propylene glycol, or polyethylene glycol. When administered in liquid form, the pharmaceutical composition(s) particularly contains from about 0.005 to about 95% by weight of the active substance. For example, a dose of about 10 mg to about 1000 mg once or twice a day could be administered orally.

In another embodiment, the composition(s) of the present disclosure can be tableted with conventional tablet bases such as lactose, sucrose, and cornstarch in combination with binders, such as acacia, cornstarch, or gelatin, disintegrating agents such as potato starch or alginic acid, and a lubricant such as stearic acid or magnesium stearate. Liquid preparations are prepared by dissolving the composition(s) in an aqueous or non-aqueous pharmaceutically acceptable solvent which may also contain suspending agents, sweetening agents, flavoring agents, and preservative agents as are known in the art.

For parenteral administration, for example, the composition(s) may be dissolved in a physiologically acceptable pharmaceutical carrier and administered as either a solution or a suspension. Illustrative of suitable pharmaceutical carriers are water, saline, dextrose solutions, fructose solutions, ethanol, or oils of animal, vegetative, or synthetic origin. The pharmaceutical carrier may also contain preservatives and buffers as are known in the art.

When a therapeutically effective amount of the composition(s) is administered by intravenous, cutaneous, or subcutaneous injection, the compound is particularly in the form of a pyrogen-free, parenterally acceptable aqueous solution or suspension. The preparation of such parenterally acceptable solutions, having due regard to pH, isotonicity, stability, and the like, is well within the skill in the art. A particular pharmaceutical composition for intravenous, cutaneous, or subcutaneous injection may contain, in addition to the active agent(s), an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition(s) of the present disclosure may also contain stabilizers, preservatives, buffers, antioxidants, or other additives known to those of skill in the art.

As noted, particular amounts and modes of administration can be determined by one skilled in the art. One skilled in the art of preparing formulations can readily select the proper form and mode of administration, depending upon the particular characteristics of the composition(s) selected, the infection to be treated, the stage of the infection, and other relevant circumstances using formulation technology known in the art, described, for example, in Remington: The Science and Practice of Pharmacy, 22^{nd} ed.

Additional pharmaceutical methods may be employed to control the duration of action of the composition(s). Increased half-life and/or controlled release preparations may be achieved through the use of polymers to conjugate, complex with, and/or absorb the active substances described herein. The controlled delivery and/or increased half-life may be achieved by selecting appropriate macromolecules (for example but not by way of limitation, polysaccharides, polyesters, polyamino acids, homopolymers polyvinyl pyrrolidone, ethylenevinylacetate, methylcellulose, or carboxymethylcellulose, and acrylamides such as N-(2-hydroxypropyl) methacrylamide), and the appropriate concentration of macromolecules as well as the methods of incorporation, in order to control release. The compound(s) may also be ionically or covalently conjugated to the macromolecules described above.

Another possible method useful in controlling the duration of action of the composition(s) by controlled release preparations and half-life is incorporation of the composition(s) or functional derivatives thereof into particles of a polymeric material such as polyesters, polyamides, polyamino acids, hydrogels, poly(lactic acid), ethylene vinylacetate copolymers, copolymer micelles of, for example, polyethylene glycol (PEG) and poly(l-aspartamide).

Examples of bacterial families which contain bacterial species against which the presently disclosed compositions and treatment protocols are effective include, but are not limited to: Alicyclobacillaceae, Bacillaceae, Listeriaceae, Paenibacillaceae, Pasteuriaceae, Planococcaceae, Sporolactobacillaceae, Staphylococcaceae, Thermoactinomycetaceae, Aerococcaceae, Carnobacteriaceae, Enterococcaceae, Lactobacillaceae, Leuconostocaceae, Streptococcaceae, Caldicoprobacteraceae, Christensenellaceae, Clostridiaceae, Defluviitaleaceae, Eubacteriaceae, Graciibacteraceae, Heliobacteriaceae, Lachnospiraceae, Oscillospiraceae, Peptococcaceae, Peptostreptococcaceae, Ruminococcaceae, Syntrophomonadaceae, Veillonellaceae, Halanaerobiaceae, Halobacteroidaceae, Natranaerobiaceae, Thermoanaerobacteraceae, and Thermodesulfobiaceae.

Specific bacteria that can be treated with the compositions and methods of the present disclosure include, but are not limited to: Enterococcus faecalis, Enterococcus faecium, Staphylococcus aureus, Methicillin-resistant Staphylococcus aureus (MRSA), Staphylococcus epidermidis, Methicillin-resistant Staphylococcus epidermidis (MRSE), oxacillin-resistant Staphylococcus aureus (ORSA), vancomycin-resistant Staphylococcus aureus (VRSA), Streptococcus pneumonia, e.g., penicillin-resistant Streptococcus pneumonia, Streptococcus mutans, Streptococcus sanguinis, Bacillus anthracis, Bacillus cereus, Clostridium botulinum, and Listeria monocytogenes.

In certain embodiments, the compositions of the present disclosure may be provided as a package or kit, which include, for example, substantially pure preparations of the active agents described herein, combined with pharmaceutically acceptable carriers, diluents, solvents, excipients, and/or vehicles to produce an appropriate pharmaceutical composition. One embodiment of such a package or kit therefore includes at least one container with an antibiotic and at least one container with a potentiating compound. Each container may comprise a pharmaceutically acceptable carrier, diluent, solvent, excipient, and/or vehicle. Each container may comprise one or more doses of the antibiotic and/or of the potentiating compound. The package or kit may comprise a plurality of containers with an antibiotic and a potentiating compound. The package or kit may comprise a plurality of containers each with a different an antibiotic and a plurality of containers with the same potentiating compound or different potentiating compounds. The package or kit may further comprise a set of directions for administering the antibiotic(s) and potentiating compound(s).

### EXAMPLES

The inventive concepts of the present disclosure will now be discussed in terms of several specific, non-limiting, examples. The examples described below, which include particular embodiments, will serve to illustrate the practice of the present disclosure, it being understood that the particulars shown are by way of example and for purposes of illustrative discussion of particular embodiments of the present disclosure only and are presented in the cause of providing what is believed to be a useful and readily understood description of procedures as well as of the principles and conceptual aspects of the inventive concepts.

In certain embodiments of the present disclosure, BPEI with an average molecular weight of 0.5 kDa was investigated for in vitro antimicrobial activity, and in certain embodiments synergy, with various antibiotics, including β-lactams, against MRSA. The results demonstrated that MRSA was re-sensitized to β-lactams at previously ineffective levels by the addition of BPEI. Likewise, a dramatic synergistic effect was seen with chloramphenicol, a drug that is used rarely in humans due to harmful side effects. However, BPEI did not show synergy with vancomycin or novobiocin antibiotics. Additionally, it was demonstrated that BPEI interacts with the cell wall of MRSA using laser scanning confocal microscopy (LSCM) images taken with BPEI conjugated to AlexaFluor 488, a fluorescent molecule. Without wishing to be bound by theory, these studies, using whole cells of MRSA, indicate a possible mode of action for BPEI's antimicrobial and synergistic properties that involves binding to the cell wall. PBP2a, located in the cell wall, is inactivated by antibiotics that prevent the synthesis of anionic teichoic acid polymers required for proper localization of the PBP2a enzyme. This function is in contrast to the common paradigm of directly targeting the enzyme. If BPEI binds to the cell wall, it may interrupt the function of teichoic acids, inactivate PBP2a and restore β-lactam activity. The cytotoxicity of BPEI was investigated using in vitro assays with murine fibroblast cells, revealing a low toxicity towards mammalian cells at concentrations resulting in antibiotic synergy. Lastly, the effectiveness of ampicillin towards non-resistant S. aureus was not affected by BPEI. These results indicate a dramatic benefit to human health, for example by using an ampicillin-BPEI combination, as a routine antibiotic therapy, to eliminate S. aureus infections while simultaneously preventing the growth of ampicillin-resistant bacteria.

Materials and Methods

Materials

The bacteria used in this work were obtained from the American Type Culture Collection (methicillin-resistant Staphylococcus aureus (MRSA) strain ATCC 700787, Staphylococcus aureus ATCC 25923, Escherichia coli ATCC 11775, Bacillus subtilis (Ehrenberg) Cohn ATCC 23059). Chemicals from Sigma-Aldrich (e.g., DMSO, ampicillin, methicillin, vancomycin, chloramphenicol, and novobiocin) were used as purchased.

Preparation and Characterization of Cationic Polymers

Branched and linear polyethylenimine was obtained from Sigma-Aldrich, Inc. as high molecular weight (˜25 kDa) or low molecular weight (˜0.5 kDa) polymers. The molecule has polycationic character from the protonation of its amine functional groups based on its protonation constant (pKₐ). Protonation constants for branched PEI molecules have been reported to be around 4.5 for primary amines, 6.7 for secondary amines, and 11.6 for tertiary amines.²⁰ Thus, at pH=7.2, the BPEI has many positively-charged primary amines to interact with teichoic acid.

Growth Inhibition Assays

Compounds were tested against methicillin-resistant Staphylococcus aureus (MRSA) strain ATCC 700787, which also exhibits reduced susceptibility to vancomycin. A stock culture was diluted in tryptic soy broth (TSB) and delivered into pre-sterilized 96-well plates. Stock solutions of BPEI and antibiotics, prepared in DMSO, were added to each well of a 96-well plate (final DMSO concentration was 1%), followed by inoculation with MRSA in TSB. Optical density measurements were performed with a Tecan Infinite M200 plate reader and an initial OD₆₀₀ value recorded. The plates were incubated for 20 h in a humidified incubator at 37° C. Plates were removed, orbitally shaken, and a final OD₆₀₀ value recorded. The final OD₆₀₀ reading was subtracted from the initial OD₆₀₀ reading to obtain the change in OD₆₀₀ (recorded in the figures as ΔOD₆₀₀). Antimicrobial activity was determined by the change in optical density. Duplicate measurements were performed and the average reported.

Using the average ΔOD₆₀₀ values, separate MIC values for BPEI and ampicillin were determined by the lowest concentration of each that inhibited growth. From this, fractional inhibitory concentration indices (FIC) were calculated for all wells that showed inhibition.^{21,22}

Cytotoxicity Assay

Mammalian cell cytotoxicity assays were performed on NIH/3T3 mouse fibroblast cells by adding 5,000 cells per well into 96-well plates. The cells were allowed to adhere overnight at 37° C. in a humidified incubator (5% CO₂ atmosphere). Test compounds were diluted in DMSO and added to the wells so that the final concentration of DMSO per well did not exceed 1% by volume. The plates containing treated and control cells were incubated for 48 hours and cell viability was determined by MTT assay.²³ Duplicate measurements were performed and the average reported.

Synthesis of the BPEI:Dye Conjugate

Low-molecular weight BPEI (Sigma-Aldrich) was added to Alexa Fluor 488 dye provided in the Alexa Fluor 488 Protein Labeling Kit (Life Technologies) at a ratio of 200 μL BPEI (3 mg/mL stock in Milli-Q H₂O) per tube of powdered dye. After allowing the dye and BPEI to form the conjugate over 1.5 h at 25° C., the product was stored at 4° C. and used without further purification.

Labeling MRSA Cells with the BPEI:Dye Conjugate

Cultures of E. coli ATCC 11775 and methicillin-resistant S. aureus ATCC 700787 at mid-log growth phase were pelleted by centrifugation at 2,000×g for 5 minutes at room temperature, and the growth media supernatant was removed. 6 μM DAPI in phosphate-buffered saline (1×PBS, pH 7.2) was added to resuspend the cell pellet, which was allowed to incubate for 5 minutes at room temperature. The BPEI-dye conjugate was then added to a final concentration of 100 μg/mL. Two control samples were prepared with either unconjugated BPEI or Alexa Fluor 488 alone and added to MRSA cells as described above. The stained bacterial cells were immediately fixed by resuspension with 4% paraformaldehyde (PFA) followed by a 10-fold dilution in 1×PBS. Cells were added to a microscope slide immediately prior to imaging.

Confocal Microscopy

PFA-fixed cells were mounted in 1×PBS and imaged using a Leica SP8 LSCM with a 63×/1.4NA oil objective. A 405 nm GaN diode laser line was used to image DAPI, and a 488 nm argon laser line was used to observe Alexa Fluor 488 fluorescence. Single optical sections were acquired of cells that had adhered to the coverslip, for highest axial resolution, with a pixel resolution of 80 nm×80 nm. Instrument settings were kept fixed for all imaging to allow for direct comparison of fluorescence intensity.

Image Processing

To visualize the relative localization of fluorescence, independent of intensity, images were processed (ImageJ v1.49m) such that the total fluorescence intensity within each image was visible (Figure S8, left column). To determine the relative fluorescence intensity between images, the fluorescence intensity of both the DAPI and the Alexa Fluor 488 were normalized to the respective intensities in the MRSA sample treated with BPEI conjugated with Alexa Fluor 488 (Figure S8, right column).

WTA Purification for NMR Studies

The NMR experiments require 1-5 mg WTA isolated from 1 L of bacterial cell culture. The poly(ribitol phosphate) WTA used in this work was isolated from B. subtilis W23^{24,25} (Bacillus subtilis (Ehrenberg) Cohn ATCC® 23059™) rather than from MRSA, allowing high volumes (500-2000 mL) of culture to be processed with minimal risk to personnel. This also allows us to take the sample into the NMR facility room which is not rated for BSL-2 work. In the rare instance of sample breakage, using WTA from B. subtilis W23 does not present a clean-up hazard nor require decontamination of the expensive NMR analysis probes, magnets, or spectrometers.

B. subtilis W23 cells were grown in LB broth to an OD₆₀₀ reading of approximately 0.8. After growth, the cells were harvested by centrifugation and physically disrupted using an Avestin® EmulsiFlex-C3 homogenizer. The insoluble cell wall was collected, placed in boiling 6% (w/v) SDS, washed with sterile water and EDTA then placed in a 10% trichloroacetic acid (TCA) treatment for 48 hours at 4° C. After allowing the TCA to remove the bound WTA from the cell wall, the WTA was collected in the supernatant and placed into a 500 Da molecular weight cutoff dialysis membrane. Membrane dialysis was performed at 4° C. in 1.5 L of sterile water with continual water changes over a 24-hour period. The final 4 h of dialysis took place in a 1 kDa molecular weight cutoff membrane to assure sample purity. The sample was lyophilized and kept at −20° C. until use.

NMR Spectroscopy

NMR samples were prepared in Eppendorf tubes by mixing teichoic acid with low-Mw BPEI in water. The pH was measured with a microscopic pH probe and adjusted to 7.2 if necessary. A 3-mm NMR tube was filled with 160 μL of a 2 mM sample of WTA/D₂O or a combination of 2 mM BPEI with 2 mM WTA in D₂O. NMR data collection use Agilent VNMRS-500 MHz equipped with a PFG probe tuned to the ³¹P resonance frequency. Data acquisition and processing were performed using VNMRJ 2.2C software on a system running Red Hat Enterprise Linux.

Results and Discussion

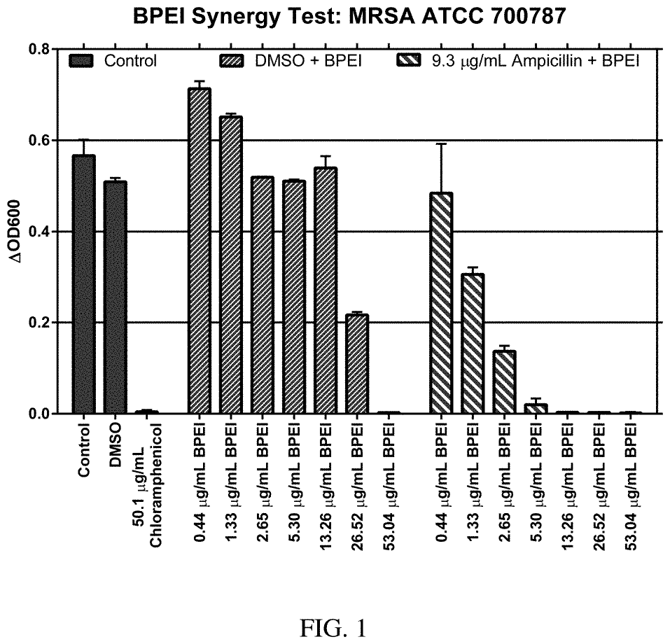

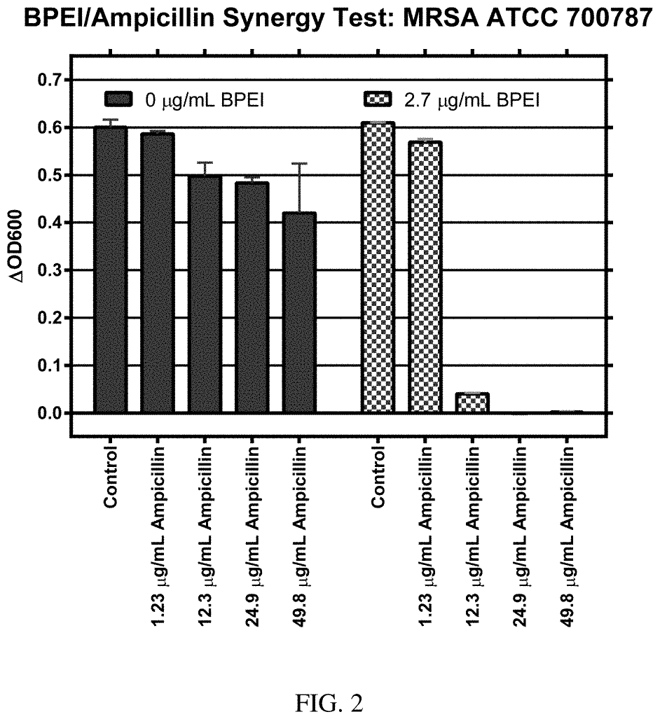

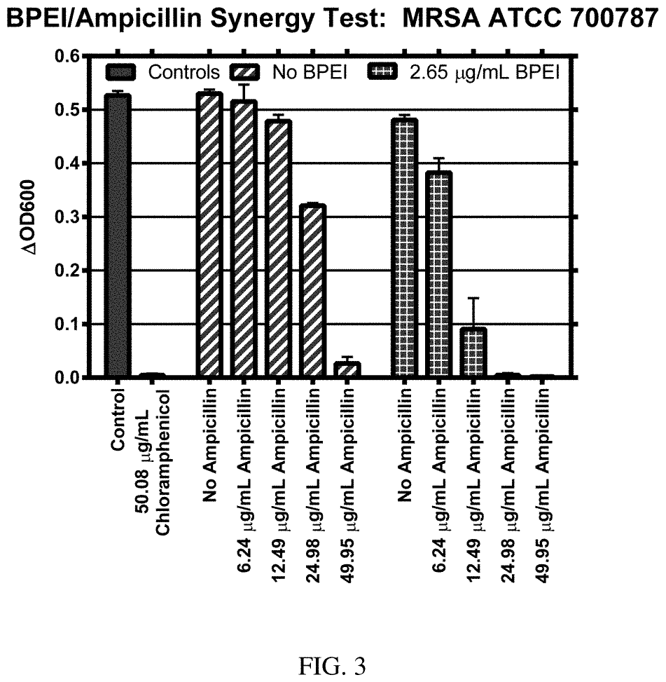

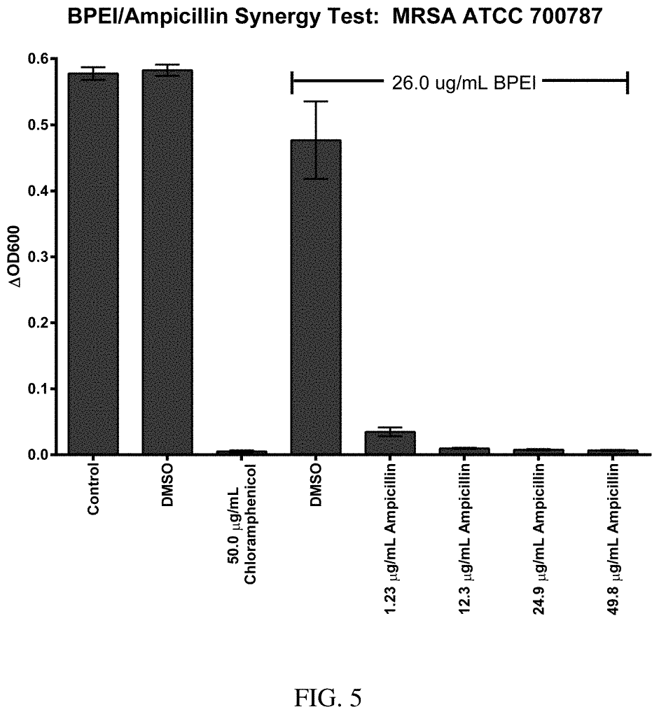

While there are numerous MRSA strains, Staphylococcus aureus subsp. aureus ATCC® 700787™ was isolated in Port Chester, N.Y. from blood culture, exhibits vancomycin intermediate resistant (VISA) and also expresses mecA to produce PBP2a. The effect of BPEI concentration on ampicillin effectiveness is shown in FIGS. 1-3, 5 and Table 1. The growth of MRSA in vitro after 20 hours of incubation was inhibited by ampicillin when the antibiotic was co-administered with low molecular-weight branched poly(ethylenimine), low-Mw BPEI. At a fixed ampicillin concentration of 9.3 μg/mL, BPEI shows synergy by inhibiting MRSA at a concentration of 5.3 μg/mL (FIG. 1). In the absence of ampicillin, BPEI itself can inhibit bacterial growth only at a concentration of 53 μg/mL, a tenfold increase (FIG. 1). As BPEI concentration is reduced, 9.3 μg/mL ampicillin becomes less able to inhibit the growth of MRSA. The ampicillin concentration was varied while the amount of BPEI was fixed at 2.7 μg/mL. This BPEI concentration, as observed in FIG. 1, produced attenuated but measurable growth in conjunction with 9.3 μg/mL ampicillin. As shown in FIG. 2, the minimum inhibitory concentration (MIC) is between 12-25 μg/mL ampicillin using 2.7 μg/mL of BPEI, and that ampicillin without added BPEI is ineffective against MRSA at or below 50 μg/mL. A second set of data confirmed that MRSA growth is slowed with 12-25 μg/mL ampicillin combined with 2.7 μg/mL BPEI yet shows that ampicillin alone does not inhibit MRSA growth at 25 μg/mL but does substantially at a higher concentration of 50 μg/mL (FIG. 3). Thus, in one embodiment the combination of ampicillin and BPEI can inhibit the growth of MRSA in combinations of either 12 μg/mL ampicillin+2.7 μg/mL BPEI or 9.3 μg/mL ampicillin+5.3 μg/mL BPEI. A further increase in BPEI (26 μg/mL) causes a substantial reduction in the MIC of ampicillin (to approximately 1.2 μg/mL, FIG. 5).

MRSA cells (ATCC 700787) were used to inoculate growth media containing ampicillin and low-Mw BPEI at 37° C. for 20 hours. Entries in Table 1 are the optical density (OD) of the growth media measured at a wavelength of 600 nm. Each entry is the average of duplicate measurements. The bold line separates combinations that allowed MRSA growth (OD₆₀₀ values above 0.05) from those combinations that prevented MRSA growth. The ampicillin MIC is 32 μg/mL demonstrating antibiotic resistance without the presence of BPEI. Values highlighted in bold text were determined to arise from synergy between ampicillin and BPEI.

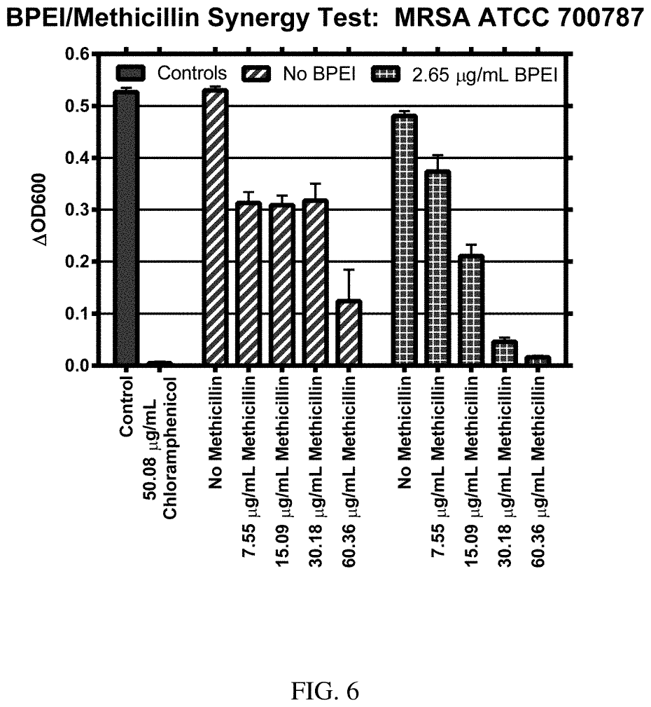

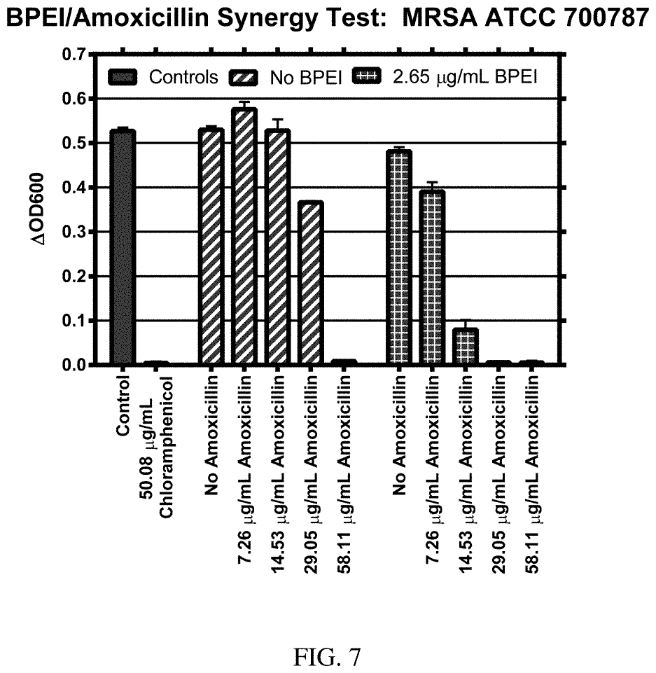

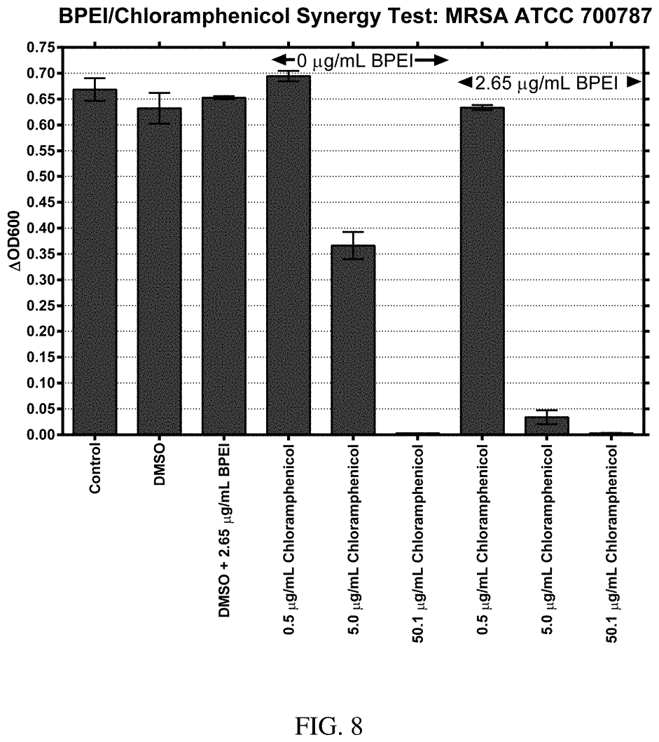

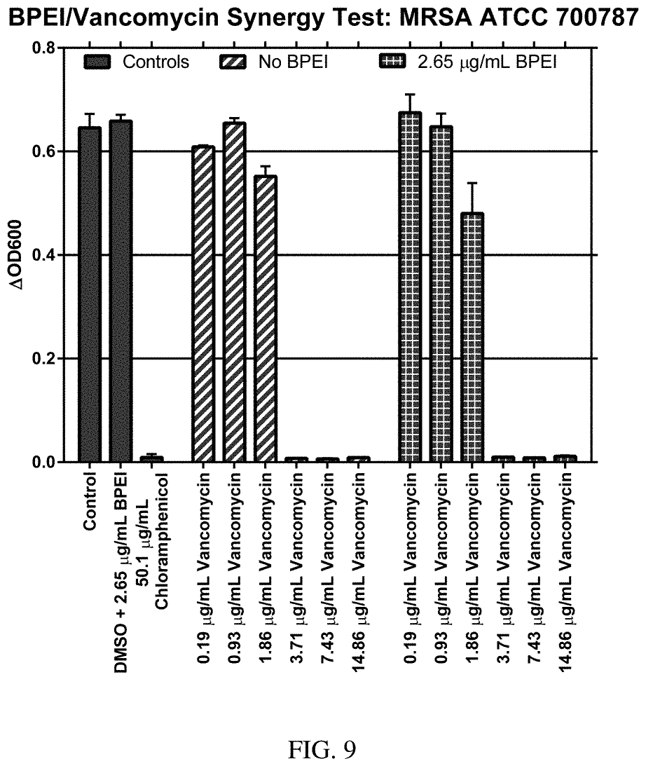

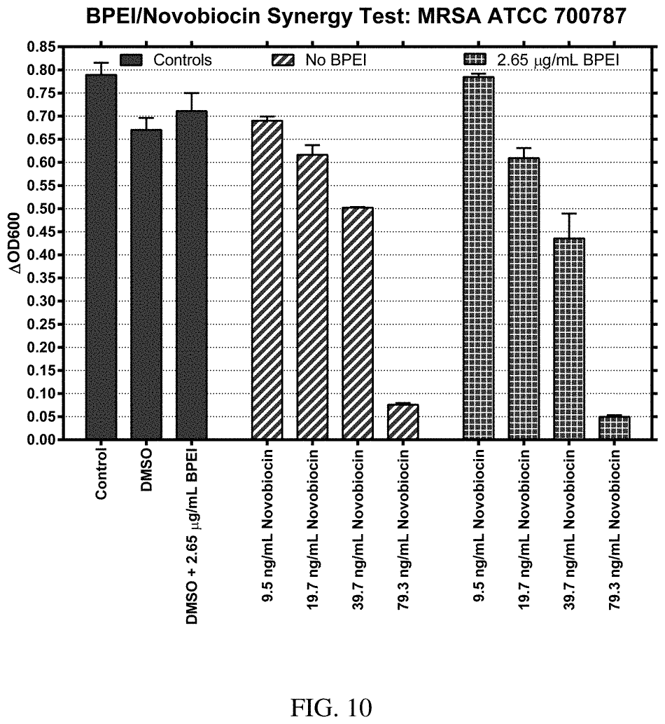

The demonstrated synergistic effect between BPEI and ampicillin indicates that similar effects can be obtained with other may be seen with other β-lactam antibiotics. For the β-lactams methicillin and amoxicillin, 30 μg/mL was able to inhibit MRSA in the presence of 2.7 μg/mL BPEI (FIG. 6 and FIG. 7, respectively). Likewise, addition of 2.7 μg/mL BPEI induced a 10-fold decrease in the MIC for chloramphenicol (FIG. 8). BPEI did not show any synergy with vancomycin or novobiocin against MRSA (FIG. 9 and FIG. 10). The three β-lactam antibiotics showed increased efficacy, as did chloramphenicol, a member of the amphenicol class. However, novobiocin, an aminocoumarin, and vancomycin, a glycopeptide, showed no increased efficacy in combination with BPEI. Aminocoumarins work by inhibiting DNA gyrase. This differs from the mechanism of action of amphenicols, which work in the bacterial ribosome to inhibit protein synthesis. Glycopeptides inhibit peptidoglycan crosslinking by binding to the peptidoglycan stems, and β-lactams occupy active sites of transpeptidase proteins that create the crosslinks between two peptide stems.

The ability of BPEI to restore ampicillin effectiveness against MRSA is shown in Table 1. MRSA shows resistance towards ampicillin, with a minimum inhibitory concentration (MIC) of 32 μg/mL, but the presence of low-Mw BPEI (16 μg/mL) rendered MRSA susceptible to ampicillin at a 32× lower dose (MIC=1 μg/mL). At a reduced BPEI concentration of 8 μg/mL, the ampicillin MIC was still decreased, but only to 8 μg/mL. In the absence of ampicillin, BPEI itself inhibited growth of MRSA at a concentration of 64 μg/mL. With these values, it is possible to calculate the fractional inhibitory concentration (FIC) index for each combination. When the FIC is equal to or below 0.5, ampicillin and low-Mw BPEI stop MRSA growth through synergistic effects. Synergy occurred between 8 μg/mL ampicillin and 8 μg/mL low-Mw BPEI (FIC=0.375), as well as with combinations of 16 μg/mL low-Mw BPEI with 1, 2, 4, and 8 μg/mL ampicillin (FIC=0.281, 0.313, 0.275, 0.500 respectively). Although MRSA does not grow in the presence of 0.5 μg/mL ampicillin and 32 μg/mL low-Mw BPEI, the FIC index is 0.516. Thus, in other embodiments, a combination which is slightly above 0.5 also inhibits the bacteria such as 0.52, 0.53, 0.54, or 0.55.

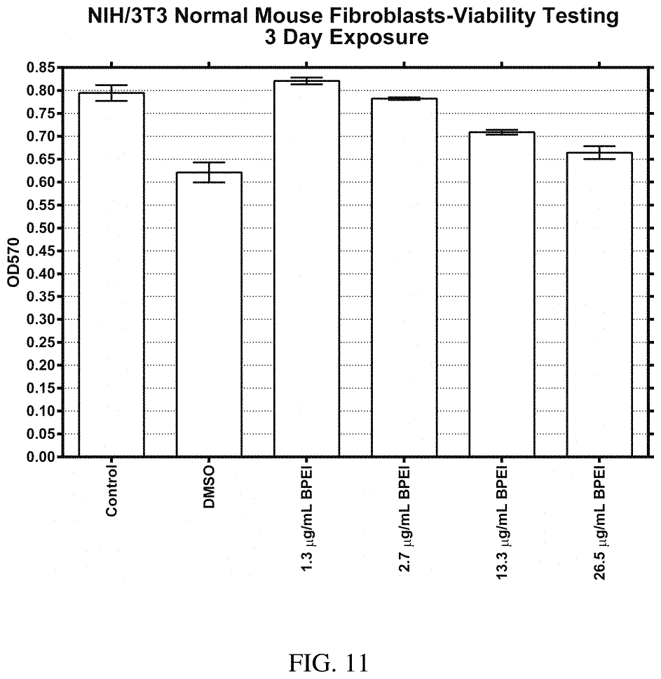

The trend of lower ampicillin MIC with higher BPEI concentration suggests that superior therapeutic treatment of MRSA may occur as BPEI levels increase. However, BPEI must demonstrate low mammalian cytotoxicity to be clinically viable. It is known that most branched PEIs are nontoxic below 25 kDa MW. To test the cytotoxicity of BPEI used in these experiments (MW˜0.5 kDa), mouse fibroblasts (NIH/3T3 cells) were incubated over three days in the presence of varying concentrations of BPEI (FIG. 11). Viability was determined using the MTT assay, following the protocol of Hansen et al. At 2.65 μg/mL, only 1.5% of the fibroblasts became nonviable over the three-day incubation period compared against the control sample. This indicates negligible cytotoxicity from low-Mw BPEI. From the checkerboard assay (Table 1), synergy occurs with 16 μg/mL low-Mw BPEI which causes ˜10% reduction in fibroblast viability (FIG. 11). At 26.5 μg/mL low-Mw BPEI, only a 16.3% reduction in viability compared to the control sample was observed. Thus, BPEI concentrations that induce synergy are also associated with low cytotoxicity. Low-Mw BPEI is non-toxic whereas high-Mw BPEI penetrates mammalian cells leading to cytotoxicity and renal failure. Low-Mw BPEI, up to 1000 μg/mL, does not affect the membrane of human cells as judged by the lack of hemolysis in red blood cells and the lack of lactate hydrogenase leakage from HEp-2 cells.³⁰ The BPEI concentration for this toxicity testing is ˜25 times larger than the highest amount, 64 μg/mL, used for antibiotic potentiation.

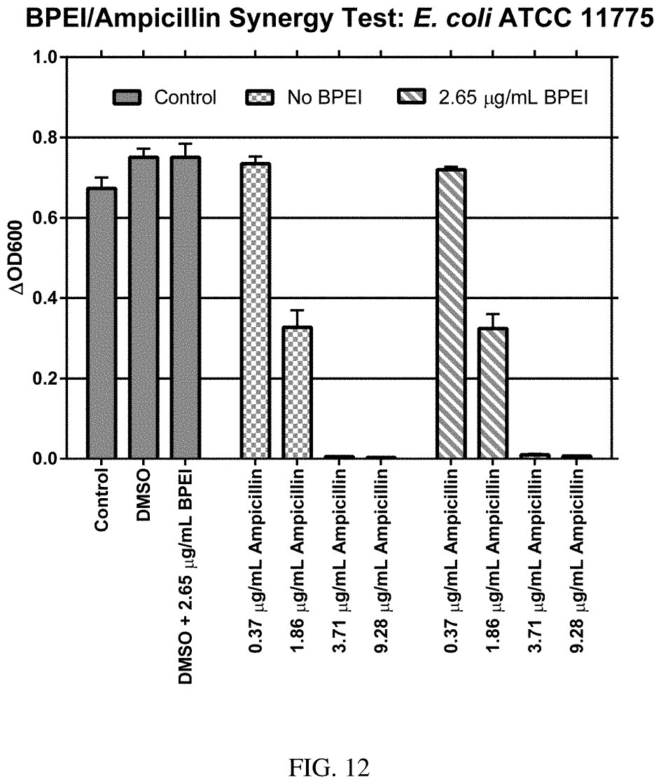

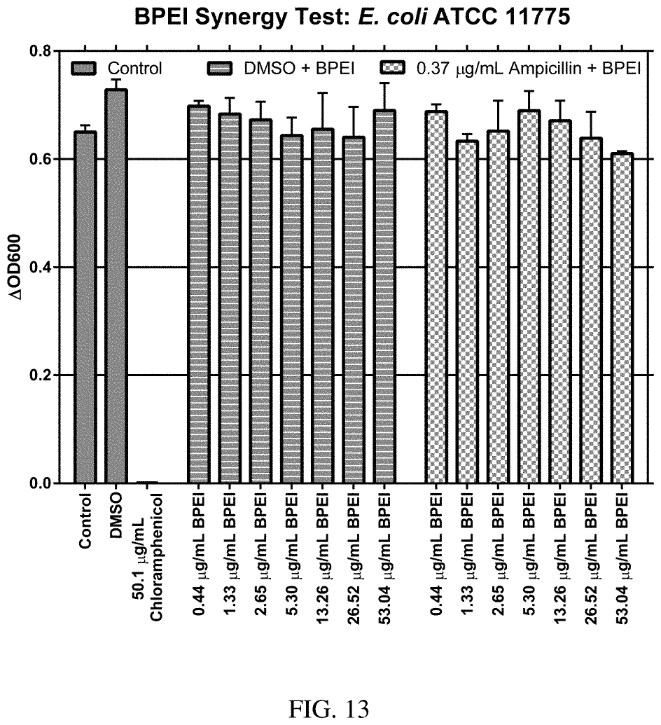

Antibiotic potentiation by BPEI does not extend to Gram-negative Escherichia coli (ATCC 11775). Results of growing E. coli in the presence of ampicillin alone and with a fixed BPEI concentration (2.65 μg/mL) are shown in FIG. 12. FIG. 13 shows the result of testing BPEI with a fixed ampicillin concentration (0.37 μg/mL). In the first data set (FIG. 12), the observed ampicillin MIC was identical at 3.7 μg/mL with or without BPEI addition. In the second data set (FIG. 13), BPEI did not show inhibitory effects on growth of E. coli up to 53 μg/mL, either by itself or with 0.37 μg/mL ampicillin. This is in contrast to the MRSA data in which the ampicillin MIC decreased by a factor of 32 when the BPEI concentration was 16 μg/mL. Additionally, our data differs from previously reported results showing PEI-induced antibiotic synergy against Gram-negative bacteria, including E. coli and P. aeruginosa when using a high-Mw LPEI (insteas of the low-Mw BPEI used herein.

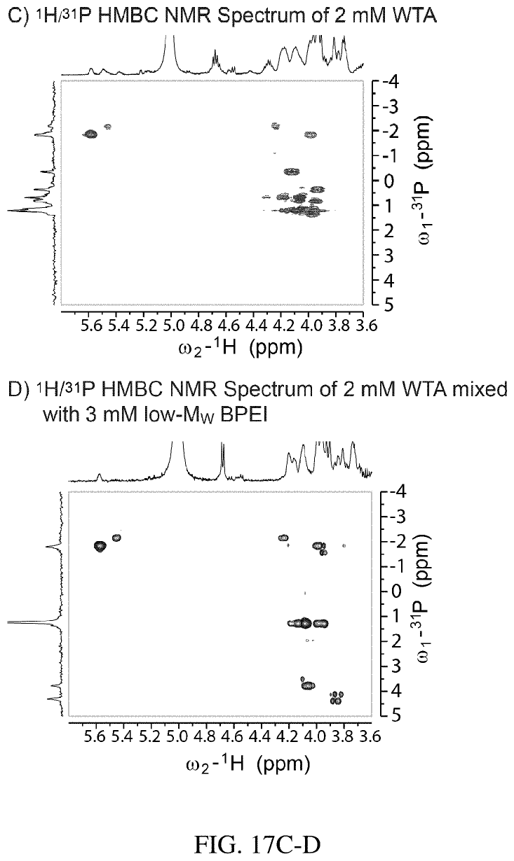

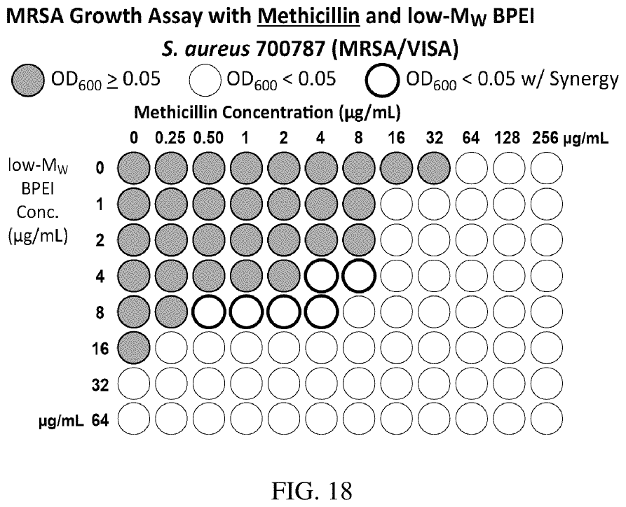

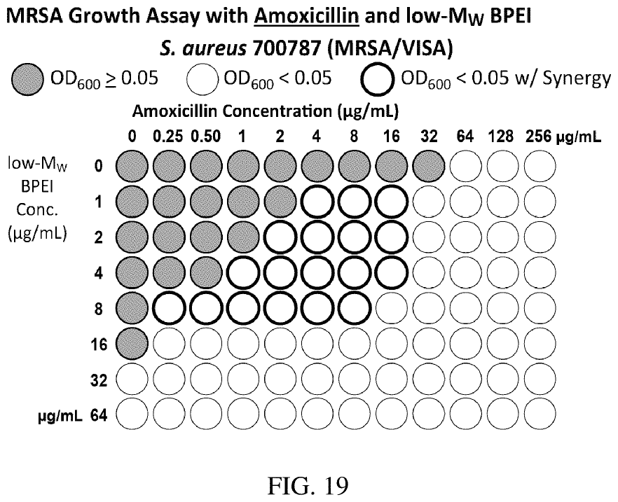

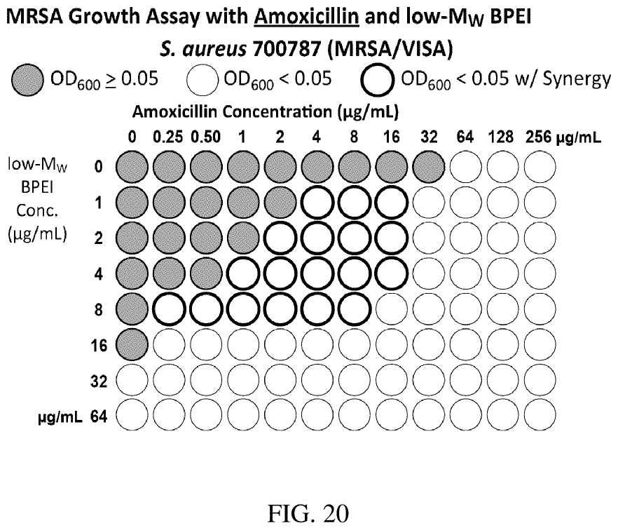

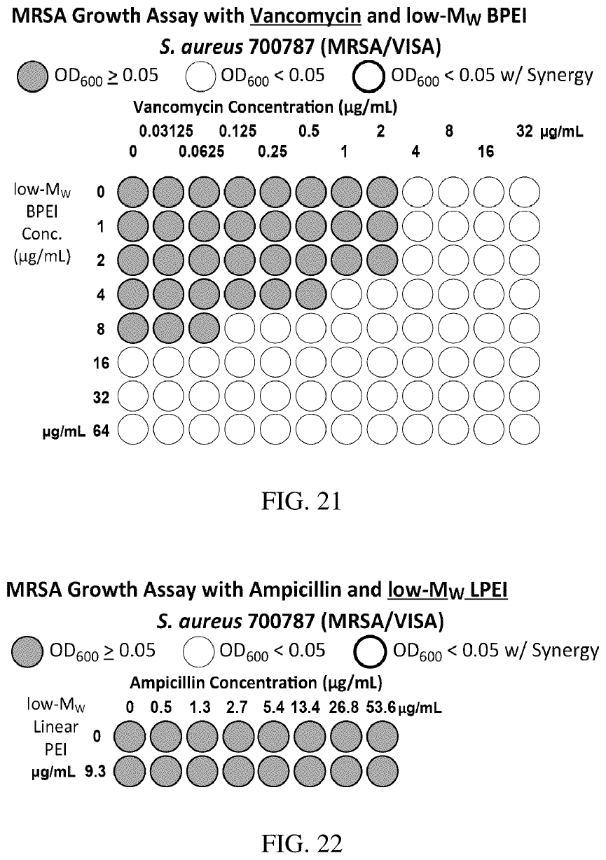

Checkerboard assays were performed which confirmed anti-MRSA effectiveness of several β-lactam antibiotics, including methicillin (FIG. 18), amoxicillin (FIG. 19), and ampicillin (FIG. 20), when administered with low-Mw BPEI. Without BPEI, MRSA is resistant (MIC >32 μg/mL) to methicillin, amoxicillin, and ampicillin. Increasing the concentration of low-Mw BPEI decreases the methicillin MIC against the MRSA strain ATCC 700787™ that is also vancomycin-intermediate resistant (VISA). Synergy occurred with at least 6 combinations that were tested. Checkerboard data for amoxicillin (FIG. 19) and ampicillin (FIG. 20) also showed synergistic activity for certain tested combinations. Vancomycin (D) has an MIC of 4 μg/ml, denoted as intermediate resistance, but did not exhibit synergy with BPEI (FIG. 21). Instead, the effect was additive. Linear PEI (LPEI) had no effect on efficacy (FIG. 22). LPEI has 2 primary amines at the ends of the polymer and mass is similar (0.6 kDa LPEI vs. 0.5 kDa low-Mw BPEI). Using a small data set, LPEI (0, 1.3, 2.7, 5.4, 13.4, 26.8, and 53.6 μg/mL) did not potentiate ampicillin (8 μg/mL).

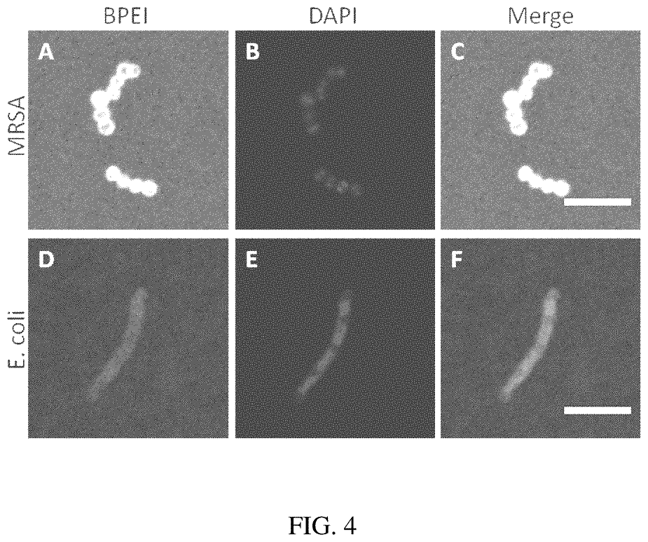

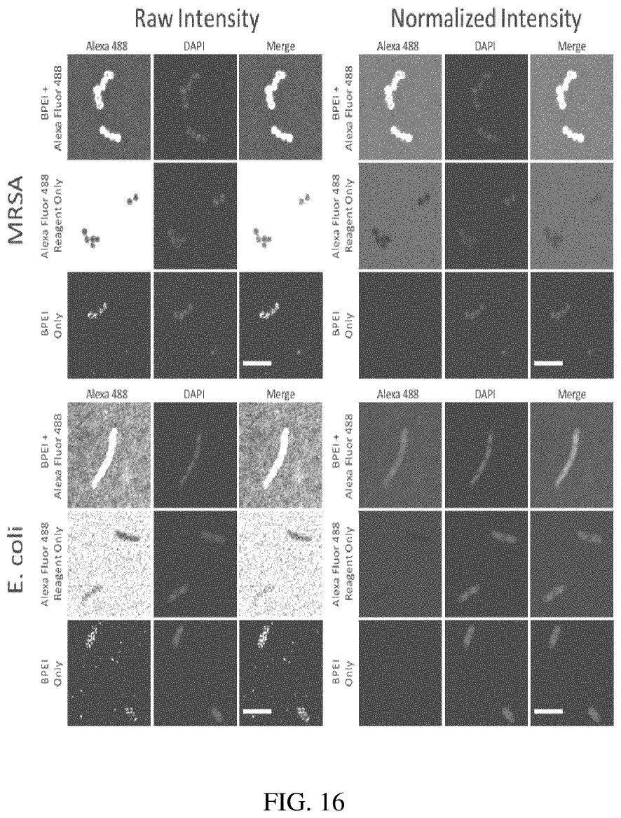

Without wishing to be bound by theory, data presented herein indicate that BPEI interacts or interferes with with PBP2a. This would prevent the enzyme from functioning properly while allowing the β-lactam to disable PBP1 and PBP3. If true, BPEI's interaction with MRSA should be confined to the cell wall. By conjugating BPEI to a fluorescent marker, Alexa-Fluor 488, we were able to visualize BPEI localization in bacterial cultures using LSCM. Individual transverse optical sections clearly show BPEI interaction with the MRSA cell wall region (FIG. 4A). Using DAPI, a DNA-binding fluorescent dye, as a marker for the cytoplasm within the cells (FIG. 4B) the merged image (FIG. 4C) confirms that BPEI was not detected within the cytoplasm, verifying that BPEI does not traverse the lipid bilayer membrane. Similar optical sections of E. coli cells treated with BPEI-conjugated Alexa-Fluor 488 revealed minimal fluorescence intensity within the cell envelope, indicating a weaker interaction between the E. coli cell envelope and BPEI (FIGS. 4D-F). This may explain the absence of antibiotic potentiation against E. coli in our present study. Additional LSCM images of the samples are provided as supplemental data (FIG. 16).

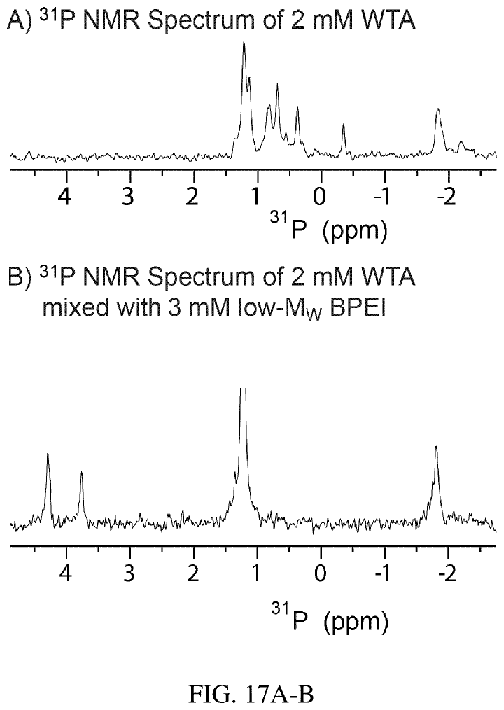

The microscopy data, showing BPEI located in the cell wall region and not the cytoplasm, indicates that the observed antibiotic potentiation against MRSA is caused by an interaction of BPEI with some component of the bacterial cell wall. One major component of the Gram-positive cell wall is wall teichoic acid (WTA), a phosphodiester polymer whose anionic phosphate groups have been shown to interact strongly with metal cations. BPEI, with its polycationic properties, has the potential for very strong electrostatic interactions with the polyanionic WTA molecules, for example via the primary amines of BPEI, and the phosphate groups of WTA. This interaction can be observed using nuclear magnetic resonance (NMR) studies of mixed BPEI-teichoic acid solutions when compared to NMR spectra of teichoic acid alone. The 1-D ³¹P spectra (FIG. 17A-B) show significant changes after mixing WTA with low-Mw BPEI. WTA is a phosphodiester polymer with heterogeneous arrangement of N-acetylglucosamine (NAG), D-alanine, and hydroxyl groups. This creates variations in conformation of the poly(ribitol) backbone and differences in the phosphate conformations that generate distinct ³¹P NMR peaks. In the presence of low-Mw BPEI, the ³¹P NMR peak at 1.3 ppm is very intense, demonstrating that a large fraction of the phosphates have similar conformations. However, signals near 4 ppm are produced by phosphates in a deshielded environment. The downfield shift arises from a loss of electron density around the phosphorous nucleus, an effect that could be caused by a hydrogen bond between the phosphate oxygen and a BPEI amine group. The addition of BPEI also increases the intensity of cross peaks in the ¹H{³¹P} HMBC (heteronuclear multiple bond coupling) NMR data (FIG. 17C-D). This experiment relies on strong through-bond coupling between the ¹H and ³¹P nuclei. For flexible molecules, internal motion and dynamics causes relaxation of NMR signals and thus the ¹H{³¹P} HMBC signals are difficult to observe. When molecular motion is restricted, the signals are stronger.

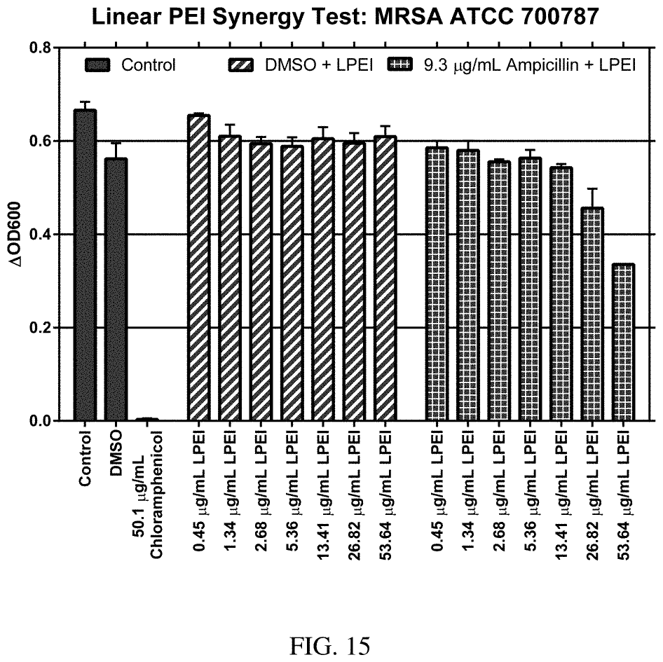

Without wishing to be bound by theory, phosphate:amine binding from the WTA:BPEI interactions likely occurs through electrostatic attraction between the numerous cationic primary amines of BPEI and anionic phosphate groups of WTA. If this assumption is correct linear PEI, with only the 2 primary amines at its terminal ends, should not affect ampicillin's MIC values. The 0.6 kDa form of LPEI (similar in mass to the 0.5 kDa BPEI) does not inhibit MRSA growth (FIG. 15) until its concentration is very high (54 μg/mL). Low molecular weight quaternary ammonium compounds have recently been shown to overcome resistance if the number of cationic sites is increased. Therefore, in at least certain embodiments, the optimal cationic amine polymer (BPEI) should have a relatively high number of primary amines with a low molecular weight to minimize cytotoxicity.

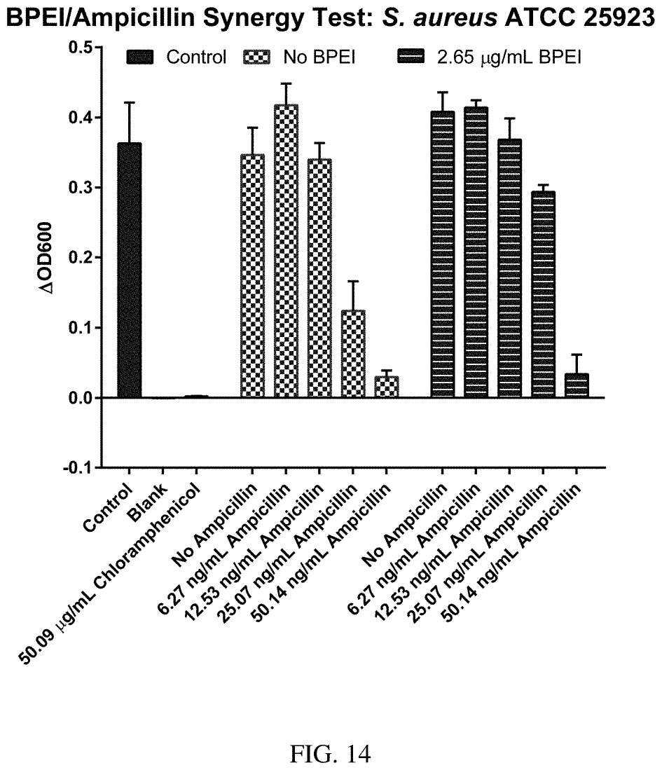

Because low-Mw BPEI binds to WTA, the cationic polymer has the ability to change WTA properties by altering molecular structure and/or creating steric bulk from the branched BPEI. This would change, interfere with, or prevent, the interaction of WTA with PBP2a and thus disable the enzyme. The same effect can be created through WTA-deficient strains of MRSA, which become re-sensitized to amoxicillin, ampicillin, methicillin, nafcillin, and ceftizoxime. An inhibitor of WTA synthesis, tunicamycin, re-sensitizes MRSA to β-lactams such as methicillin, oxacillin, cefotaxime, and several others. Inhibition of another regulatory gene, tarG, also re-sensitizes MRSA strains to traditional β-lactams like imipenem. Thus WTA, while apparently not essential to viability, is involved in β-lactam resistance. WTA helps to optimally localize PBP2a, and WTA-deficient mutants show a decreased functionality of the protein. It additionally localizes PBP4, which is essential for the highly cross-linked peptidoglycan exhibited by MRSA and for the expression of β-lactam resistance in community-acquired strains. Thus, restoration of β-lactam activity in therapeutic clinical usage could be achieved with antibiotics or other compounds that target WTA synthesis or interrupt the ability of WTA to localize PBP2a in the proper configuration required for peptidoglycan crosslinking. If this perspective is true, there should be little or no benefit when BPEI and ampicillin are used to treat non-resistant S. aureus strains that do not express PBP2a. Results in FIG. 14 show that the ampicillin MIC against methicillin-susceptible S. aureus ATCC 25923 was about 50 ng/mL. When combined with 2.65 μg/mL of BPEI, the ampicillin MIC of S. aureus ATCC 25923 was not reduced.

These data provide additional evidence for the interruption by BPEI of the ability of teichoic acid to locate PBP2/2a and 4 in the proper orientation. Further, the data for S. aureus ATCC 25923 indicate a treatment method which also prevent antibiotic resistant infections. By using a combination of BPEI and ampicillin to treat a non-resistant S. aureus infection, the emergence of β-lactam resistant strains in vivo would be slowed. This benefit would not be possible by treatment with the antibiotic alone.

Rather than developing new inhibitors which require exhaustive clinical testing, as the results indicate, we have identified FDA-approved β-lactam antibiotics that can regain their previously-lost efficacy against MRSA. Such β-lactam-BPEI combination formulations provide dramatic benefits to human health when used as a routine antibiotic therapy, eliminating, for example, S. aureus infections while simultaneously preventing the growth of ampicillin-resistant bacteria. Further, the data for S. aureus ATCC 25923 indicate a route to treat, and prevent, antibiotic resistant infections. By using a combination of BPEI and ampicillin (or other β-lactams) to treat a non-resistant S. aureus infection, the emergence of β-lactam resistant strains in vivo can be slowed. This benefit would not be possible with ampicillin alone.

Combination treatments of an antibiotic with a compound that blocks the resistance pathway have previously been shown to be a viable therapeutic strategy. For example, β-lactam antibiotics can be deactivated by bacteria that possess β-lactamases. The increasing incidence of β-lactamases has been problematic since the 1960s. Researchers discovered that amoxicillin could be co-administered with the β-lactamase inhibitor, clavulanic acid. In such combination therapies, commercialized as Augmentin®, the antibiotic is not deactivated, but rather can successfully inhibit cell wall synthesis and kill the bacterium. The amoxicillin and clavulanic acid treatment provides a compelling precedent demonstrating that combination therapies can be a successful antibacterial approach. While this approach using the Augmentin® combination effectively treats methicillin-susceptible S. aureus (MSSA) infections, MRSA bacteria will endure absent new therapies. The approach described in the present disclosure can be used to simultaneously eliminate both MSSA and MRSA infections to limit tissue damage from toxins, decreasing morbidity and mortality. Patients will not have to endure multiple treatments with an array of antibiotics to clear the infection, thereby improving quality of life. Fewer medical complications and courses of treatment will result in better patient outcomes at a lower cost to patients and providers.

Additionally, the large synergy demonstrated with chloramphenicol, while impressive, is tempered by the knowledge that chloramphenicol is rarely used clinically in humans because of adverse side effects. However, bone marrow suppression, a common side effect induced by chloramphenicol, is dose-dependent, which indicates that by lowering the MIC by using the synergistic BPEI:chloramphenicol compositions described herein can make human treatments with chloramphenicol clinically relevant once more.

The present disclosure includes in at least one embodiment an antibiotic composition comprising a β-lactam antibiotic, and a branched poly(ethylenimine) (BPEI) compound. The antibiotic composition may be effective against a bacterium which is resistant to the β-lactam antibiotic when the β-lactam antibiotic is administered alone. The β-lactam antibiotic and the BPEI compound of any of said antibiotic compositions may interact synergistically against the resistant bacterium. Any of said antibiotic compositions may have a synergistic fractional inhibitory concentration (FIC) against the resistant bacterium. Said FIC may be 0.5. The BPEI compound of any of said compositions may have a molecular weight in a range of 0.1 kilodalton (kDa) to 25 kDa. Any of said antibiotic compositions may comprise a pharmaceutically-acceptable carrier, vehicle, or diluent. Any of said antibiotic compositions may be effective against at least one resistant bacterium selected from the group consisting of Enterococcus faecalis, Enterococcus faecium, Staphylococcus aureus, methicillin-resistant Staphylococcus aureus (MRSA), Staphylococcus epidermidis, Methicillin-resistant Staphylococcus epidermidis (MRSE), oxacillin-resistant Staphylococcus aureus (ORSA), vancomycin-resistant Staphylococcus aureus (VRSA), a Streptococcus pneumonia, Streptococcus mutans, Streptococcus sanguinis, Bacillus anthracis, Bacillus cereus, Clostridium botulinum, and Listeria monocytogenes.

The present disclosure includes in at least one embodiment a method of treating a bacterial infection in a subject, comprising administering to the subject a cephem β-lactam antibiotic and a branched poly(ethylenimine) (BPEI) compound (non-pegylated or pegylated) in amounts effective in inhibiting the bacterial infection in the subject. In the method the bacterial infection may be caused by a β-lactam antibiotic-resistant bacterium. In any of the methods the β-lactam antibiotic and the BPEI compound may be administered to the subject in sequential or simultaneous steps, or as a composition comprising both the β-lactam antibiotic and the BPEI compound. In any of said methods the β-lactam antibiotic and the BPEI compound may inhibit the bacterial infection by interacting synergistically against the bacterium causing the bacterial infection. In any of said methods the β-lactam antibiotic and the BPEI compound together may have a synergistic fractional inhibitory concentration (FIC) against the bacterium causing the bacterial infection. In said methods the FIC may be ≤0.5. In any of said methods the BPEI compound may have a molecular weight in a range of 0.1 kilodalton (kDa) to 25 kDa. In any of said methods the bacterial infection may be caused by a bacterium selected from the group consisting of Enterococcus faecalis, Enterococcus faecium, Staphylococcus aureus, methicillin-resistant Staphylococcus aureus (MRSA), Staphylococcus epidermidis, methicillin-resistant Staphylococcus epidermidis (MRSE), oxacillin-resistant Staphylococcus aureus (ORSA), vancomycin-resistant Staphylococcus aureus (VRSA), a Streptococcus pneumonia, Streptococcus mutans, Streptococcus sanguinis, Bacillus anthracis, Bacillus cereus, Clostridium botulinum, and Listeria monocytogenes. In non-limiting embodiments the the cephem β-lactam antibiotic may comprise cephem, cephalosporin C, cefoxitin, cephalosporin, cephamycin, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline, or be selected from the group consisting of cephem, cephalosporin C, cefoxitin, cephalosporin, cephamycin, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline. In any of said methods the cephem β-lactam antibiotic and the BPEI compound may be administered to the subject in sequential or simultaneous steps, or as a composition comprising both the β-lactam antibiotic and the BPEI compound. In one non-limiting embodiment, the present disclosure is directed to a method for treating a bacterial infection in a subject by administering to the subject in need of such treatment an antibacterially-effective combination of a β-lactam antibiotic and a BPEI compound, wherein the antibacterially-effective combination has a fractional inhibitory concentration (FIC)≤0.5.

It will be understood from the foregoing description that various modifications and changes may be made in the various embodiments of the present disclosure without departing from their true spirit. The description provided herein is intended for purposes of illustration only and is not intended to be construed in a limiting sense. Thus, while embodiments of the present disclosure have been described herein so that aspects thereof may be more fully understood and appreciated, it is not intended that the present disclosure be limited to these particular embodiments. On the contrary, it is intended that all alternatives, modifications and equivalents are included within the scope of the inventive concepts as defined herein. Thus the examples described above, which include particular embodiments, will serve to illustrate the practice of the present disclosure, it being understood that the particulars shown are by way of example and for purposes of illustrative discussion of particular embodiments only and are presented in the cause of providing what is believed to be a useful and readily understood description of procedures as well as of the principles and conceptual aspects of the inventive concepts. Changes may be made in the formulations and compositions described herein, the methods described herein or in the steps or the sequence of steps of the methods described herein without departing from the spirit and scope of the present disclosure.

What is claimed is:

1. A method of treating a bacterial infection in a subject in need of such treatment, comprising:
administering to the subject a cephem β-lactam antibiotic and a branched poly(ethylenimine) (BPEI) compound in amounts effective in inhibiting the bacterial infection in the subject, wherein the amounts of the cephem β-lactam antibiotic and the BPEI compound inhibit the bacterial infection by interacting synergistically against the bacterium causing the bacterial infection, wherein the bacterium is selected from the group consisting of Enterococcus faecalis, Enterococcus faecium, methicillin-resistant Staphylococcus aureus (MRSA), methicillin-resistant Staphylococcus epidermidis (MRSE), oxacillin-resistant Staphylococcus aureus (ORSA), vancomycin-resistant Staphylococcus aureus (VRSA), a Streptococcus pneumonia, Streptococcus mutans, Streptococcus sanguinis, Bacillus anthracis, Bacillus cereus, Clostridium botulinum, and Listeria monocytogenes, and wherein the amounts of the cephem β-lactam antibiotic and BPEI compound have a cephem β-lactam antibiotic:BPEI compound mass ratio in a range of 100:1 to 1:100, and wherein the cephem β-lactam antibiotic and BPEI are optionally combined with a a pharmaceutically-acceptable carrier, vehicle, or diluent.

2. The method of claim 1, wherein the cephem β-lactam antibiotic is selected from the group consisting of cephem, cephalosporin C, cefoxitin, cephalosporin, cephamycin, cefazolin, cephalexin, cephalothin, cefaclor, cefamandole, cefuroxime, cefotetan, cefoxitin, cefixime, cefotaxime, cefpodoxime, ceftazidime, ceftriaxone, cefepime, cefpirome, and ceftaroline.

3. The method of claim 1, wherein the wherein the bacterium is further selected from the group consisting of methicillin-resistant Staphylococcus aureus (MRSA), oxacillin-resistant Staphylococcus aureus (ORSA), vancomycin-resistant Staphylococcus aureus (VRSA), and methicillin-resistant Staphylococcus epidermidis (MRSE).

4. The method of claim 1, wherein the bacterium is a methicillin-resistant Staphylococcus aureus (MRSA).

5. The method of claim 1, wherein the bacterium is an oxacillin-resistant Staphylococcus aureus (ORSA).

6. The method of claim 1, wherein the bacterium is a vancomycin-resistant Staphylococcus aureus (VRSA).

7. The method of claim 1, wherein the bacterium is a methicillin-resistant Staphylococcus epidermidis (MRSE).

8. The method of claim 1, wherein the cephem β-lactam antibiotic and the BPEI compound are administered to the subject in sequential or simultaneous steps, or as a composition comprising both the cephem β-lactam antibiotic and the BPEI compound.

9. The method of claim 1, wherein the BPEI compound has a molecular weight in a range of 0.1 kilodalton (kDa) to 25 kDa.

10. The method of claim 1, wherein the BPEI compound is pegylated.

11. The method of claim 1, wherein the cephem β-lactam antibiotic:BPEI compound mass ratio is in a range of 50:1 to 1:50.

12. The method of claim 1, wherein the cephem β-lactam antibiotic and the BPEI compound are provided in an antibacterially-effective combination having a fractional inhibitory concentration (FIC) 0.5.
